(19) United States
(12) Patent Application Publication
(10) Pub. No.: US 2018/0333461 A1
(43) Pub. Date: Nov. 22, 2018

(54) METHODS AND COMPOSITIONS FOR TREATING OR PREVENTING FLAVIVIRUS INFECTIONS

(71) Applicant: THE UNIVERSITY OF QUEENSLAND, St. Lucia (AU)

(72) Inventor: Paul Robert YOUNG, Ashgrove (AU)

(21) Appl. No.: 15/534,023

(22) PCT Filed: Dec. 8, 2014
(86) PCT No.: PCT/AU2014/050403

§ 371 Date: Jun. 8, 2017

(51) Int. Cl. A61K 38/20; A61K 39/12

(52) U.S. Cl. CPC A61K 38/20; A61K 39/12

(57) ABSTRACT

Disclosed are methods and compositions for the treatment or prevention of Flavivirus infections. In particular, the present invention discloses TLR4 antagonists for use in treating or preventing disease associated with Flavivirus infections, including Dengue virus infections.

### FIELD OF THE INVENTION

This invention relates generally to methods and compositions for the treatment or prevention of Flavivirus infections. In particular, the invention relates to TLR4 antagonists for use in treating or preventing disease associated with Flavivirus infections, including Dengue virus infections.

### BACKGROUND OF THE INVENTION

Dengue virus infection is an increasing problem in tropical and subtropical areas, and is endemic in over a hundred countries (1). With increased international travel and climate change extending the range of the mosquito vector, more populations will become increasingly at risk of dengue infection. Recent estimates suggest there are approximately 390 million dengue infections globally per year, 96 million of these being symptomatic and with many thousands of deaths (1). Despite this global health burden, no vaccine or therapeutic intervention has yet been licensed. Dengue infection causes a spectrum of diseases ranging from undifferentiated fever, mild classical dengue fever (DF) to severe and potentially fatal dengue hemorrhagic fever (DHF) and dengue shock syndrome (DSS). Severe disease is characterized by the rapid onset of capillary leak accompanied by thrombocytopenia and altered hemostasis. While the mechanistic basis of dengue mediated capillary leak remains the subject of considerable conjecture, the host immune response appears to be the primary determinant of disease outcome. DHF/DSS patients have higher levels of circulating pro-inflammatory cytokines and chemokines, leading to the suggestion that it is these vasoactive mediators that play a direct and leading role in the collapse of vascular integrity (2,3). Although DHF and DSS can accompany primary infection, they more frequently occur upon secondary infection with a heterologous dengue virus strain. The anamnestic induction of high levels of cross-reactive, but sub-neutralizing antibodies during the acute stage of secondary infection is thought to contribute to antibody-dependent enhancement (ADE) of infection in Fc receptor-bearing cells resulting in higher virus production (4, 5). In addition, cross-reactive, and consequently low affinity T-cell responses to the secondary infecting virus results in delayed viral clearance (6) and a skewing of the cytokine response that elevates inflammatory cytokines and contributes to the pathology of severe disease (7). Other factors including viral strain virulence, host gene polymorphism, and nutritional status may also play roles as risk factors in DHF/DSS development (8).

The Dengue virus non-structural protein NS1 is a multi-functional, 48-55 kDa glycoprotein, that is initially synthesized as a soluble monomer and becomes membrane-associated following dimerization in the lumen of the endoplasmic reticulum (9). The recent crystal structure determination of NS1 has revealed exposed hydrophobic domains on the dimeric form that are likely to mediate this membrane-association (10). Intracellular NS1 participates in early viral RNA replication and is found in virus-induced vesicular compartments that house the viral replication complexes (11). NS1 is also transported to the cell surface, where it either remains associated with the cell membrane, or is secreted as a soluble, lipid-associated hexameric species. A small subset of cell-surface expressed NS1 has been shown to be membrane-associated via a glycosylphosphatidylinositol (GPI)-anchor, which can mediate signal transduction on specific antibody engagement, thereby facilitating enhanced production of viral progeny (12). Secreted NS1 (sNS1) can be detected in the bloodstream of infected patients from the first day of symptoms and may circulate at remarkably high levels (up to 50 μg/mL) during the acute phase of infection (13). This early viral biomarker of infection has now become a target for routine diagnosis (14, 15) with levels shown to correlate with viremia and disease severity in secondary Dengue virus infection (13, 16), NS1 has been proposed as playing a number of roles in the pathology of infection, including both stimulation and inhibition of complement pathways, and damage to platelets and endothelial cells by cross-reactive anti-NS1 antibodies (9).

### SUMMARY OF THE INVENTION

The present invention arises in part from the determination that sNS1 elicits pro-inflammatory cytokine production via TLR4 signaling. This finding identifies sNS1 as a dengue virus-encoded pathogen-associated molecular pattern. In addition, it has been determined that sNS1 increases permeability of human dermal endothelial cell monolayers in an in vitro model of vascular leakage. The striking similarities in cellular responses to LPS and NS1 via TLR4 suggest that sNS1 is a viral counterpart of bacterial endotoxin. Moreover, the present inventors have found that inhibition of TLR4 by TLR4 antagonists prevents NS1-mediated cytokine release, thereby providing a strategy for therapeutic intervention in viral disease. Of significanc, sNS1 is conserved across the Flavivirus genus of arboviruses and it is proposed therefore that TLR4 antagonists are useful for treating Flavivirus infections generally.

The present inventors have reduced the above findings to practice in methods and compositions for modulating production of pro-inflammatory mediators by immune cells and/or vascular leakage in subjects with Flavivirus infections and for treating or preventing Flavivirus infections or a symptom thereof, as described hereafter.

Accordingly, in one aspect, the present invention provides methods for modulating production of a pro-inflammatory mediator (e.g., a cytokine) by a cell (e.g., an immune cell (e.g., a macrophage or monocyte), or an endothelial cell) in a subject with a Flavivirus infection. These methods generally comprise, consist or consist essentially of contacting the cell with a pro-inflammatory mediator-modulating amount of a TLR4 antagonist.

Another aspect of the present invention provides methods for modulating vascular leakage in a subject with a Flav/virus infection. These methods generally comprise, consist or consist essentially of contacting an endothelial cell with a vascular leakage-modulating amount of a TLR4 antagonist.

In a related aspect, the present invention provides methods for inhibiting the binding of sNS1 to a cell (e.g., an immune cell or an endothelial cell) that mediates disease symptoms (e.g., production of a pro-inflammatory mediator, vascular leakage, etc.) associated with a Flavivirus infection. These methods generally comprise, consist or consist essentially of contacting the cell with a sNS1-binding-inhibiting amount of a TLR4 antagonist.

In another aspect, the present invention provides methods for modulating vascular leakage in a subject with a Flavivirus infection. These methods generally comprise, consist or consist essentially of administering to the subject a vascular leakage-modulating amount of a TLR4 antagonist.

Yet another aspect of the present invention provides methods for treating or preventing a Flavivirus infection or a symptom thereof in a subject. These methods generally comprise, consist or consist essentially of administering an effective amount of a TLR4 antagonist to the subject.

In related aspects, the present invention provides the use of a TLR4 antagonist for modulating, production of a pro-inflammatory mediator (e.g., a cytokine) by a cell (e.g., an immune cell or an endothelial cell), which is associated with a Flavivirus infection, or for modulating vascular leakage that is associated with a Flavivirus infection, or for inhibiting the binding of sNS1 to a cell that mediates disease symptoms associated with a Flavivirus infection, or for treating or preventing a Flaviviridae virus infection. In some embodiments, the TLR4 antagonist is manufactured as a medicament for any one or more of those applications.

Suitably, the Flavivirus is a virus selected from the group consisting of Dengue virus (DENV), Japanese encephalitis virus (JBI), Yellow fever virus (YFV), Murray Valley encephalitis virus (MVEV), West Nile virus (WNV), Tick-borne encephalitis virus (TBEV), St Louis encephalitis virus (SLEV), Alfuy virus (AV), Koutango virus (KV), Cacipacore virus (CV), and Yaounde virus (YV). In specific embodiments, the Flavivirus is selected from Dengue virus serotype I, II, III, or IV. In some embodiments, the methods further comprise identifying that the subject has or is at risk of developing a Flavivirus infection, suitably prior to administration of the TLR4 antagonist. In illustrative examples of this type, the methods comprise determining the presence of NS1 (e.g., soluble or non-soluble forms of NS1) in the subject (e.g., in a biological sample of the subject, illustrative examples of which include blood, serum, plasma, saliva, cerebrospinal fluid, urine, skin or other tissues, or fractions thereof), suitably prior to administration of the TLR4 antagonist.

Non-limiting examples of suitable TLR4 antagonists include nucleic acids, peptides, polypeptides, peptidomimetics, carbohydrates, lipids or other organic (carbon containing) or inorganic molecules. In specific embodiments, the TLR4 antagonist is selected from liposaccharide compounds, carbohydrates, small molecule inhibitors, nucleic acid molecules (e.g., ones that inhibit the transcription or translation of a TLR4 gene or that mediate RNA interference), decoy receptors and antagonist antibodies. In some embodiments, the TLR4 antagonist reduces the expression of the TLR4 gene or the level or functional activity (e.g., reduces the level of a TLR4 polypeptide, reduces TLR4 mediated cytokine production and/or antagonizes the TLR4 signaling pathway) of a TLR4 expression product to less than about 9/10, ⅘, 7/10, ⅗, ½, ⅖, 3/10, ⅕, 1/10, 1/20, 1/50, 10⁻¹, 10⁻², 10⁻, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴ or about 10⁻¹⁵ of the expression of the TLR4 gene, or the level or functional activity of a corresponding TLR4 expression product in the absence of the TLR4 antagonist. In some embodiments, the TLR4 antagonist is a selective TLR4 antagonist. In other embodiments, the TLR4 antagonist is a non-selective TLR4 antagonist.

The TLR4 antagonist may be administered alone or in combination with one or more ancillary agents that treat or ameliorate the symptoms of a Flavivirus infection. Accordingly, in still another aspect, the present invention provides pharmaceutical compositions, suitably for treating a Flavivirus infection or symptom thereof. These compositions comprise, consist or consist essentially of a TLR4 antagonist and an ancillary anti-Flaviviridae virus agent, optionally together with a pharmaceutically acceptable carrier or diluent.

In a related aspect, the present invention provides methods for treating or preventing a Flavivirus infection or symptom thereof in a subject. These methods generally comprise, consist or consist essentially of administering concurrently to the subject an effective amount of a TLR4 antagonist and an effective amount of an ancillary anti-Flavivirus agent. Suitably, the TLR4 antagonist and the ancillary anti-Flavivirus agent are administered in synergistically effective amounts.

In specific embodiments, the ancillary anti-Flavivirus agent is selected from interferons, illustrative examples of which include interferon alpha (e.g., interferon alpha 2a and interferon alpha 2b) and interferon beta (e.g., interferon beta 1a and interferon beta 1b), or nucleic acid constructs from which the ancillary anti-Flavivirus agent is expressible.

In another related aspect, the present invention provides the use of a TLR4 antagonist and an ancillary anti-Flavivirus agent for treating or preventing a Flavivirus infection or symptom thereof. In some embodiments, the TLR4 antagonist and the ancillary anti-Flavivirus virus agent are manufactured as a medicament for this application. Suitably, the TLR4 antagonist and the ancillary anti-Flavivirus agent are formulated for concurrent administration.

### BRIEF DESCRIPTION OF THE DRAWINGS

FIG. 1 is a graphical representation showing that Dengue NS1 activates human and mouse innate immune responses. (A) NS1 induces IL-6 secretion by human PBMC. Cells were treated for 24 hrs. with LPS (100 ng/mL), NS1 (40 μg/mL), or an analogous NS1 sample immune-depleted with anti-NS1 or anti-E antibody. IL-6 was measured by ELISA. Data shown is mean±SD of three donors. The inset immunoblot is of the supernatant fraction following immuno-depletion using anti-NS1 antibody or isotype-matched control antibody. (B) NS1 and LPS activate NF-κB-dependent transcription in a mouse macrophage cell line expressing GFP under the control of the ELAM promoter, Cells were treated with NS1 (5, 10, 20, 40 80, 160, 400 μg/mL) or LPS (0.0064, 0.032, 0.16, 0.8, 4, 20, 100 ng/mL). Results show expression of GFP relative to the mean fluorescence index of untreated cells. Data are the mean±SD from 4 independent experiments. (C) NS1 immuno-depletion prevents NF-κB-dependent promoter activity. ELAM promoter activity was monitored as in B, in response to LPS (100 ng/mL), NS1 (40 μg/mL), NS1 immuno-depleted using anti-NS1 antibody or isotype-matched control antibody (confirmed in western blot inset). Data show mean±SD of 3 independent experiments.

FIG. 2 is a graphical representation showing that the LPS-binding antibiotic polymyxin B does not affect the response to NS1. (A) Polymyxin B does not block NS1-induced IL-6 in human PBMC. NS1 and LPS were pre-incubated for 30 minutes with or without polymyxin B, then added to PBMC and incubated for 24 hours. The final concentration of polymyxin B was 25 μg/mL, with varying concentrations of LPS (0.1, 1, 10 ng/mL) and NS1 (5, 10, 20 and 40 μg/mL). Released IL-6 was measured by ELISA. (B) Polymyxin B does not block NS1-induced loss of surface CSF1R on BMMs. NS1 and LPS were pre-incubated for 30 minutes with or without polymyxin B then added to BMMs that had been starved of CSF1 overnight. The final concentration of polymyxin B was 25 μg/mL, with LPS (10-fold dilutions from 100 ng/mL) and NS1 (2′-fold dilutions from 10 μg/mL). After 1 hour of treatment, CSF1R levels were determined by flow cytometry, and the % of cells with high levels of receptor are shown. Data are the mean±SD from three (A) or five (B) independent experiments.

FIG. 3 is a graphical representation showing that NS1 from several sources has immunostimulatory activity. (A) NS1 immuno-depletion prevents the down-modulation of surface CSF1R by S2 cell-expressed NS1. BMMs starved of CSF1 overnight were treated with either nothing, CSF1 (10⁴ U/mL), LPS (100 ng/mL), NS1 (10 μg/mL), or NS1 immuno-depleted with anti-NS1 antibody or control anti-E antibody. Data are the mean±SD from three independent experiments. (B) Minimally processed CHO cell-expressed NS1 has similar activity to S2 cell-derived NS1. CHO cells were transfected with either empty expression vector, or a plasmid encoding NS1 under the control of the CMV promoter. Culture medium concentrated with 100 kDa cut-off, from untransfected cells (control SN), empty vector (pcDNA SN) or NS1 expression vector transfected cells (NS1 SN), was applied to CSF1-starved BMM. The final concentration of 5 μg/mL NS1 (as quantified by capture-ELISA) represents approximately 4-fold higher levels than produced in the CHO cell medium. Cells were also exposed to NS1 SN following immune-depletion with anti-NS1 antibody or control anti-E antibody, and to CSF1. Data are the mean SD from three independent experiments. Immunoblot analysis for NS1 confirms immuno-depletion.

FIG. 4 is a graphical representation showing that induction of TNF-α and IL-6 mRNAs in C57BL/6 BMMs and human PBMCs in response to 52-derived NS1 protein. (A) Dose-dependent production of cytokine mRNAs from treated murine BMMs. BMMs were incubated with purified NS1 (1.25, 2.5, 5, 10, 20 and 40 μg/mL) or LPS (0.1, 1, 10 and 100 ng/mL) and harvested after 3 hours. Expression of individual mRNAs were measured by real time PCR and expressed relative to hprt mRNA. (B) Time course of BMM response to LPS and NS1. BMM were incubated with either no additions (control), NS1 (40 μg/mL) or LPS (1 ng/mL) for the indicated times. Cytokine mRNAs were measured as described in panel A. A control sample is included for each time point, but generally cannot be seen on this scale. (C) Time course of PBMC response to LPS and NS1. PBMCs were treated with no additions (control), NS1 (1014/mL) or 195 (10 ng/mL) for the indicated times. Cytokine mRNAs were measured by real time PCR and expressed relative to hprt mRNA. Data are mean±range from two independent experiments (A and B) or mean±SD from four donors (C).

FIG. 5 is a graphical representation showing that NS1 activates cells via TLR4. (A) Down-modulation of BMM cell surface CSF1R by NS1 requires TLR4. CSF1R on wild-type C57BL/6 BMM (WT), tlr4^{-/-} and MyD88^{-/-}/trif^{-/-} BMMs was measured after 1 hour treatment with CSF1 (10⁴ U/mL), LPS (100 ng/mL) and NS1 (10 μg/mL). (B) Down-modulation of BMM cell surface CSF1R by NS1 does not require TLR2. BALB/c wild-type BMM (WT) and tlr2^{-/-} BMM were treated as in A or with Pam₃CSK₄ (100 ng/mL) as a TLR2 stimulus. (C-D) NS1-induction of TNF-α and IL-18 mRNAs requires TLR4. Wild-type (C57BL/6) and tlr4^{-/-} BMMs were incubated with 10 mg/mL NS1, 1 ng/mL LPS or 100 ng/′mL Pam₃CSK₄ for 3 hours, and mRNA expression determined using real time PCR relative to hprt mRNA. (E-F) NS1 induction of cytokine mRNAs does not require TLR2. Wild-type (BALB/c) and tlr2^{-/-} BMM were treated as in (C). (G-H) HEK293 cells expressing human TLR4 and MD-2 (G), but not TLR2 (H) respond to 3 hours treatment with NS1 (10 erg/mL), with induction of IL-8 mRNA, measured by real time PCR relative to hprt mRNA, and normalized to the control sample. LPS (10 ng/mL) and Pam₃CSK₄ (100 ng/mL) provide control stimuli for TLR4 and TLR2 respectively. Data are mean SD of three independent experiments (A, G, H), or mean±range from two independent experiments (B to F).

FIG. 6 is a graphical representation showing that TLR4 antagonists block NS1 activity in vitro. (A) NS1-induced IL-6 production by PBMC was inhibited by LPS-RS. Cells were pre-incubated with LPS-RS (10 μg/mL) for 30 min and subsequently stimulated with LPS (100 ng/mL) NS1 (10 ng/mL) or Pam₃CSK₄ (500 ng/mL) for 24 hours. IL-6 was assessed by capture-ELISA. (B) Anti-TLR4 antibody reduces the response to NS1. PBMC were pre-incubated with 2 μg/mL and 10 μg/mL anti-TLR4 for 1 hour and then stimulated with NS1 and LPS for 24 hours. IL-6 was assessed by capture-ELISA. Data are mean±SD for four donors (A and B). (C) NS1 induces permeability of HMEC-1 monolayers. Confluent cells in transwells were pre-treated with or without LPS-RS (10 μg/mL) for 30 minutes, and then treated with LPS (100 ng/mL) or NS1 (10 μg/mL) for 2 hours. Transfer of 4 kDa FITC-dextran across the monolayer was assessed for a further hour. Mean responses from 4 independent experiments are shown with connecting lines designating paired LPS-RS treated and untreated samples. Significance was determined by one-tailed paired t-tests; *p<0.02, ****p<0.0001.

FIG. 7 is a photographic representation showing co-localization of NS1 and TLR4 on adherent PBMCs, PBMCs were allowed to adhere to coverslips for two hours and were then treated with 10 μg/mL NS1 for 45 min, prior to fixation. Cells were stained for cell surface TLR4 (red) and NS1 (green) without permeabilization. (A) Orthographic and planar projections were obtained by confocal microscopy. In the right hand panel regions of co-localization appear as yellow. Single color controls showed no bleed between fluorescence channels. There was no background staining with secondary antibodies alone, and the secondary antibodies were demonstrated to be specific for the species of primary antibody. (B) NS1 binding correlates with TLR4 expression in the mixed PBMC population. Cells were stained as per panel A.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when ail such agents are administered as a single composition. By “simultaneously” is meant that the agents are administered at substantially the same time, and desirably together in the same formulation. By “contemporaneously” it is meant that the agents are administered closely in time, one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “agent” or “modulatory agent” includes a chemical compound, a mixture of chemical compounds, a biological macromolecule, an extract made from biological materials, a biological organism or part thereof, or other material, which induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The term “antagonist” is used in its broadest sense, and includes any compound that inhibits or abrogates the TLR4 signaling pathway. For example, an antagonist may compete with an agonist or partial agonist for binding to a receptor, thereby inhibiting the action of an agonist or partial agonist on the receptor or may inhibit or abrogate dimerization of TLR4 and block or reduce signaling through TLR4. Thus, the term “Toll like receptor 4 antagonist” or “TLR4 antagonist” refers to an agent that incapable of substantially reducing, inhibiting, blocking, and/or mitigating the activation of the TLR4 signaling pathway of a cell, for example by a TLR4 agonist or partial agonist. Inhibition of TLR4 activation by an antagonist suitably reduces or inhibits the TLR4 signaling pathway including the production of pro-inflammatory mediators including pro-inflammatory cytokines by the cell or the modulation of other cellular elements that are associated with Flavivirus disease symptoms.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present, By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “alkyl” refers to a straight chain, branched or cyclic saturated hydrocarbon group having 1 to 10 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methyl butyl, n-hexyl, 2-methyl pentyl, 3-methyl pentyl, 4-methylpentyl, 5-methylpentyl, 2-ethylbutyl, 3-ethylbutyl, heptyl, octyl, nonyl, decyl, cyclopropyl, cyclobutyl, cyclopentyl, cyclohexyl and cycloheptyl.

As used herein, the term “alkenyl” refers to a straight-chain, branched or cyclic hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, cyclopentenyl, cyclohexenyl and cyclohexadienyl.

“Aralkyl” means alkyl as defined above which is substituted with an aryl group as defined above, e.g., —CH₂phenyl, —(CH₂)₂phenyl-(CH₂)₃phenyl, —H₂CH(CH₃)CH₂phenyl, and the like and derivatives thereof.

As used herein, “aromatic” or “aryl” is intended to mean any stable monocyclic or bicyclic carbon ring of up to 7 atoms in each ring, wherein at least one ring is aromatic. Examples of such aryl elements include, but are not limited to, phenyl, naphthyl, tetrahydronaphthyl, indanyl, biphenyl, phenanthryl, anthryl or acenaphthyl.

In certain instances, substituents may be defined with a range of carbons that includes zero, such as (C₀-C₆)alkylene-aryl. If aryl is taken to be phenyl, this definition would include phenyl itself as well as, for example, —CH₂Ph, —CH₂CH₂Ph, CH(CH₃)CH₂CH(CH₃)Ph.

It will also be recognized that the compounds described herein may possess asymmetric centers and are therefore capable of existing in more than one stereoisomeric form. The invention thus also relates to compounds in substantially pure isomeric form at one or more asymmetric centers e.g., greater than about 90% ee, such as about 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

The term “antibody” herein is used in the broadest sense and specifically covers monoclonal antibodies, polyclonal antibodies, multispecific antibodies (e.g., bispecific antibodies), antibody fragments, or any other antigen-binding molecule so long as they exhibit the desired biological activity.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target (e.g., a target antigen), wherein the target-binding polypeptide sequence was obtained, by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et at, Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al. (1991) Nature 352:624-628; Marks et al. (1991)J. Mol. Biol. 222:581-597; Sidhu et al. (2004) J. Mol. Biol. 338(2):299-310; Lee et al. (2004) 3. Mol. 340(5):1073-1093; Fellouse (2004) Proc. Nat. Acad. Sci. USA 101(34):12467-12472; and Lee et al. (2004) J. Immunol. Methods 284(1-2):119-132, and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al. (1993) Proc. Natl. Acad. Sci. USA 90:2551; Jakobovits et al, (1993) Nature 362:255-258; Bruggemann et al, (1993) Year in Immuno. 7:33; U.S. Pat. Nos. 5,545,806; 5,569,825; 5,591,669 (all of GenPharm); U.S. Pat. No. 5,545,807; WO 1997/17852; U.S. Pat. Nos. 5,545,807; 5,545,806; 5,569,825; 5,625,126; 5,633,425; and 5,661,016; Marks et al. (1992) Bio/Technology 10: 779-783; Lonberg et al, (1994) Nature 368: 856-859; Morrison (1994) Nature, 368: 812-813; Fishwild et al. (1996) Nature Biotechnology 14: 845-851; Neuberger (1996) Nature Biotechnology 14: 826; and Lonberg and Huszar (1995) Intern. Rev. Immunol, 13: 65-93).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81:6851-6855). Chimeric antibodies of interest herein include “primatized” antibodies comprising variable domain antigen-binding sequences derived from a non-human primate (e.g., Old World Monkey, Ape etc.) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the Fps are those of a human immunoglobulin sequence. The humanized antibody optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. 2:593-596.

“Antibody fragments” comprise a portion of an intact antibody, suitably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

An antibody “that binds” an antigen of interest (e.g., a toll-like receptor such as TLR4) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined by fluorescence activated cell sorting (FACS) analysis or radioimmunoprecipitation (RIA), In specific, embodiments, the antibody antagonizes a receptor antigen (e.g., a toll-like receptor such as TLR4) to which it binds and in accordance with the present invention inhibits signaling, through that receptor. With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding, can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

By “corresponds to” or “corresponding to” is meant a nucleic acid sequence that displays substantial sequence identity to a reference nucleic acid sequence (e.g., at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g., at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

The term “derivatize,” “derivatizing” and the like refer to producing, or obtaining a compound from another substance by chemical reaction, e.g., by adding one or more reactive groups to the compound by reacting the compound with a functional group-adding reagent, etc.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. Non-limiting symptoms of Flavivirus infections include acute febrile illness, malaise, headache, flushing, diarrhea, nausea, vomiting, abdominal pain, myalgias and, in severe disease, production of pro-inflammatory mediators, including pro-inflammatory cytokines and vascular leakage.

The term “endothelial cell” as used herein refers to cells that line the inside surfaces of body cavities, blood vessels, and lymph vessels and making up the endothelium. Endothelial cells are typically but not necessarily thin, flattened cells.

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only.

By “expression vector” is meant any genetic element capable of directing the transcription of a polynucleotide contained within the vector and suitably the synthesis of a peptide or polypeptide encoded by the polynucleotide. Such expression vectors are known to practitioners in the art.

As used herein, the term “function” refers to a biological, enzymatic, or therapeutic function.

The term “gene” as used herein refers to any and all discrete coding regions of the cell's genome, as well as associated non-coding and regulatory regions. The term is intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

The term “group” as applied to chemical species refers to a set of atoms that forms a portion of a molecule. In some instances, a group can include two or more atoms that are bonded to one another to form a portion of a molecule. A group can be monovalent or polyvalent (e.g., bivalent) to allow bonding to one or more additional groups of a molecule. For example, a monovalent group can be envisioned as a molecule with one of its hydrogen atoms removed to allow bonding to another group of a molecule. A group can be positively or negatively charged. For example, a positively charged group can be envisioned as a neutral group with one or more protons (i.e., Fr) added, and a negatively charged group can be envisioned as a neutral group with one or more protons removed. Non-limiting examples of groups include, but are not limited to, alkyl groups, alkylene groups, alkenyl groups, alkenylene groups, alkynyl groups, alkynylene groups, aryl groups, arylene groups, iminyl groups, iminylene groups, hydride groups, halo groups, hydroxy groups, alkoxy groups, carboxy groups, thio groups, alkylthio groups, disulfide groups, cyano groups, nitro groups, amino groups, alkylamino groups, dialkylamino groups, silyl groups, and siloxy groups. Groups such as alkyl, alkenyl, alkynyl, aryl, and heterocyclyl, whether used alone or in a compound word or in the definition of a group may be optionally substituted by one or more substituents. “Optionally substituted,” as used herein, refers to a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, phenylamino, diphenylamino, benzylamino, diberizylamino, hydrazine, acyl, acylamino, diacylamino, acyloxy, heterocyclyl, heterocycloxy, heterocyclamino, haloheterocyclyl, carboxy ester, carboxy, carboxy amide, mercapto, alkylthio, benzylthio, acylthio and phosphorus-containing groups. As used herein, the term “optionally substituted” may also refer to the replacement of a CH, group with a carbonyl (C═O) group. Non-limiting examples of optional substituents include alkyl, preferably C₁₋₈ alkyl (e.g., C₁₋₆ alkyl such as methyl, ethyl, propyl, butyl, cyclopropyl, cyclobutyl, cyclopentyl or cyclohexyl), hydroxy (e.g., hydroxymethyl, hydroxyethyl, hydroxypropyl), alkoxyalkyl (e.g., methoxymethyl, methoxyethyl, methoxypropyl, ethoxymethyl, ethoxyethyl, ethoxypropyl etc.) C₁₋₈ alkoxy, (e.g., C₁₋₆ alkoxy such as methoxy, ethoxy, propoxy, butoxy, cyclopropoxy, cyclobutoxy), halo (fluoro, chloro, bromo, iodo), monofluoromethyl, monochloromethyl, monobromomethyl, difluoromethyl, dichloromethyl, dibromomethyl, trifluoromethyl, trichloromethyl, tribromomethyl, hydroxy, phenyl (which itself may be further substituted, by an optional substituent as described herein, e.g., hydroxy, halo, methyl, ethyl, propyl, butyl, methoxy, ethoxy, acetoxy, amino), benzyl (wherein the CH₂ and/or phenyl group may be further substituted as described herein), phenoxy (wherein the CH₂ and/or phenyl group may be further substituted as described herein), benzyloxy (wherein the CH₂ and/or phenyl group may be further substituted as described herein), amino, C₁₋₈ alkylamino (e.g., C₁₋₆ alkyl, such as methylamino, ethylamino, propylamino), di C₁₋₈ alkylamino (e.g., C₁₋₆ alkyl, such as dimethylamino, diethylamine, dipropylamino), acylamino (e.g., NHC(O)CH₃), phenylamino (wherein phenyl itself may be further substituted as described herein), nitro, formyl, —C(O)—C₁₋₈ alkyl (e.g., C₁₋₆ alkyl, such as acetyl), O—C(O)-alkyl (e.g., C₁₋₆ alkyl, such as acetyloxy), benzoyl (wherein the CH₂ and/or phenyl group itself may be further substituted), replacement of CH₂ with C═O, CO₂H, CO₂ C₁₋₈ alkyl (e.g., C₁₋₆ alkyl such as methyl ester, ethyl ester, propyl ester, butyl ester), CO₂phenyl (wherein phenyl itself may be further substituted), CONH₂, CONHphenyl (wherein phenyl itself may be further substituted as described herein), CONHbenzyl (wherein the CH₂ and/or phenyl group may be further substituted as described herein), CONH C₁₋₈ alkyl (e.g., C₁₋₆ alkyl such as methyl amide, ethyl amide, propyl amide, butyl amide), CONH C₁₋₈ alkylamine (e.g., C₁₋₆ alkyl such as aminomethyl amide, aminoethyl amide, aminopropyl amide, aminobutyl amide), —C(O)heterocyclyl (e.g., —C(O)-1-piperidine, —C(O)-1-piperazine, —C(O)-4-morpholine), —C(O)heteroaryl (e.g., —C(O)-1-pyridine, —C(O)-1-pyridazine —C(O)-1-pyrimidine, —C(O)-1-pyrazine), CONHdi C₁₋₈ alkyl (e.g., C₁₋₆alkyl).

“Heteroaralkyl” group means alkyl as defined above which is substituted with a heteroaryl group, e.g., —CH₂pyridinyl, —(CH₂)₂pyrimidinyl, —(CH₂)₃imidazolyl, and the like, and derivatives thereof.

The term “heteroaryl” or “heteroaromatic”, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, bezofuranyl, quinolinyl, isoquinolinyl, oxazolyl, isoxazolyl, Indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl.

Further examples of “heterocyclyl” and “heteroaryl” include, but are not limited to, the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazoyl, indolinyl, Indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyrldyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The term “heterocycle”, “heteroaliphatic” or “heterocyclyl” as used herein is intended to mean a 5-to 10-membered nonaromatic heterocycle containing from 1 to 4 heteroatoms selected from the group consisting of O, N and S, and includes bicyclic groups.

“Heterocyclylalkyl” group means alkyl as defined above which is substituted with a heterocycle group, e.g., —CH₂pyrrolidin-1-yl, —(CH₂)₂piperidin-1-yl, and the like, and derivatives thereof.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The phrase “hybridizing specifically to” and the like refer to the binding, duplexing, or hybridizing of a molecule only to a particular nucleotide sequence under stringent conditions when that sequence is present in a complex mixture (e.g., total cellular) DNA or RNA.

As used herein, the term “immune cell” refers to a cell belonging to the immune system. Immune cells include cells of hematopoetic origin such as but not limited to T lymphocytes (T cells), B lymphocytes (B cells), natural killer (NK) cells, granulocytes, neutrophils, macrophages, monocytes, dendritic cells, and specialized forms of any of the foregoing, e.g., plasmacytoid dendritic cells, Langerhans cells, plasma cells, natural killer T (NKT) cells, T helper cells, and cytotoxic T lymphocytes (CTL).

By “isolated” is meant material that is substantially or essentially free from components that normally accompany it in its native state.

The term “lower alkyl” refers to straight and branched chain alkyl groups having from 1 to 6 carbon atoms, such as methyl, ethyl, n-propyl, iso-propyl, n-butyl, tert-butyl, sec-butyl, n-pentyl, n-hexyl, 2-methylpentyl, and the like. In some embodiments, the lower alkyl group is methyl or ethyl.

As used herein, the terms “modulating”, “regulating” and their grammatical equivalents refer to an effect of altering a biological activity or effect (e.g., cytokine production, vascular leakage, TLR4 signaling etc.). For example, an agonist or antagonist of a particular biomolecule modulates the activity of that biomolecule, e.g., a receptor, by either increasing/stimulating (e.g., agonist, activator), or decreasing/inhibiting (e.g., antagonist, inhibitor) the activity or effect (e.g., cytokine production, vascular leakage, TLR4 signaling etc.) of the biomolecule, such as a receptor.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In specific embodiments, the subject is a primate such as a human. However, it will be understood that the terms “patient,” “subject,” “host” or “individual” do not imply that symptoms are present.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The terms “polynucleotide variant” and “variant” refer to polynucleotides displaying substantial sequence identity with a reference polynucleotide sequence or polynucleotides that hybridize with a reference sequence under stringent conditions as known in the art (see for example Sambrook et al., Molecular Cloning. A Laboratory Manual”, Cold Spring Harbor Press, 1989). These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally occurring allelic variants.

The terms “polypeptide,” “proteinaceous molecule,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino add, such as a chemical analogue of a corresponding naturally-occurring amino acid, as well as to naturally-occurring amino add polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. These terms also encompass polypeptides in which one or more amino adds have been added or deleted, or replaced with different amino acids.

As used herein, the terms “prevent,” “prevented,” or “preventing,” refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

The term “pro-inflammatory mediator” means an immunoregulatory agent that favors inflammation. Such agents include, cytokines such as chemokines, interleukins (IL), lymphokines, and tumor necrosis factor (TNF) as well as growth factors. In specific embodiments, the pro-inflammatory mediator is a “pro-inflammatory cytokine”. Typically, pro-inflammatory cytokines include IL-1α, IL-1β, IL-6, and TNF-α, which are largely responsible for early responses. Other pro-inflammatory mediators include LIF, IFN-γ, IFN-α, OSM, CNTF, TGF-β, GM-CSF, TWEAK, IL-11, IL-12, IL-15, IL-17, IL-18, IL-19, IL-20, IL-8, IL-16, IL-22, IL-23, IL-31, and IL-32 (Tato, et al., 2008. J. Cell 132:900; Cell 132:500, Cell 132:324). Pro-inflammatory mediators may act as endogenous pyrogens (IL-1, IL-6, TNF-α), up-regulate the synthesis of secondary mediators and pro-inflammatory cytokines by both macrophages and mesenchymal cells (including fibroblasts, epithelial and endothelial cells), stimulate the production of acute phase proteins, or attract inflammatory cells. In specific embodiments, the term “pro-inflammatory cytokine” relates to TNF-α, IL-6, IFN-β, IL-1β and IL-8.

As used herein, “racemate” refers to a mixture of enantiomers.

The terms “salts,” “derivatives” and “prodrugs” includes any pharmaceutically acceptable salt, ester, hydrate, or any other compound which, upon administration to the recipient, is capable of providing (directly or indirectly) a compound of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl, and butyl chlorides, bromides and iodides; dialkyl sulfates like dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art. For example, metal salts can be prepared by reaction of a compound of the invention with a metal hydroxide. An acid salt can be prepared by reacting an appropriate acid with a compound of the invention.

The term “selective” refers to compounds that inhibit or display antagonism towards a toll-like receptor (e.g., TLR4) without displaying substantial inhibition or antagonism towards another toll-like receptor. Accordingly, a compound that is selective for TLR4 exhibits a TLR4 selectivity of greater than about 2-fold, 5-fold, 10-fold, 20-fold, 50-fold or greater than about 100-fold with respect to inhibition or antagonism of another TLR (i.e., a TLR other than TLR4, illustrative examples of which include TLR1, TLR2, TLR3, TLR5, TLR6, TLR7, TLR8, TLR9 and TLR11).

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as defined in Table 1.

TABLE 1

| ORIGINAL RESIDUE | EXEMPLARY SUBSTITUTIONS |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology,” John Wiley & Sons Inc, 1994-1998, Chapter 15.

As used herein a “small molecule” refers to a composition that has a molecular weight of less than 3 kilodaltons (kDa), and typically less than 1.5 kilodaltons, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, less than 1.5 kilodaltons, or even less than about 1 kDa.

“Stringency” as used herein refers to the temperature and ionic strength conditions, and presence or absence of certain organic solvents, during hybridization. The higher the stringency, the higher will be the observed degree of complementarity between sequences. “Stringent conditions” as used herein refers to temperature and ionic conditions under which only polynucleotides having a high proportion of complementary bases, preferably having exact complementarity, will hybridize. The stringency required is nucleotide sequence dependent and depends upon the various components present during hybridization, and is greatly changed when nucleotide analogues are used. Generally, stringent conditions are selected to be about 10° C. to 20° C. less than the thermal melting point (Tm) for the specific sequence at a defined ionic strength and pH. The Tm is the temperature (under defined ionic strength and pH) at which 50% of a target sequence hybridizes to a complementary probe. It will be understood that a polynucleotide will hybridize to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 42° C. High stringency conditions Include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. One embodiment of high stringency conditions includes hybridizing in 6×SSC at about 45° C., followed by one or more washes in 0.2 Y SSC, 0.1% SDS at 65° C. One embodiment of very high stringency conditions includes hybridizing 0.5 M sodium phosphate, 7% SDS at 65° C., followed by one or more washes at 0.2×SSC, 1% SDS at 65° C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see CURRENT PROTOCOLS IN MOLECULAR BIOLOGY (supra) at pages 2.10.1 to 2.10.16 and MOLECULAR CLONING. A LABORATORY MANUAL (Sambrook, et al., eds.) (Cold Spring Harbor Press 1989) at sections 1.101 to 1.104.

By “substantially complementary” it is meant that an oligonucleotide or a subsequence thereof is sufficiently complementary to hybridize with a target sequence. Accordingly, the nucleotide sequence of the oligonucleotide or subsequence need not reflect the exact complementary sequence of the target sequence. In a preferred embodiment, the oligonucleotide contains no mismatches and with the target sequence.

As used herein, the terms “treatment”, “treating”, and the like, refer to obtaining a desired pharmacologic and/or physiologic effect. The effect may be therapeutic in terms of a partial or complete cure for a disease or condition (e.g., a hematologic malignancy) and/or adverse affect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition.

As used herein, underscoring or italicizing the name of a gene shall indicate the gene, in contrast to its protein product, which is indicated by the name of the gene in the absence of any underscoring or italicizing. For example, “TLR4” shall mean the TLR4 gene, whereas “TLR4” shall indicate the protein product or products generated from transcription and translation and/or alternative splicing of the “TLR4” gene.

Each embodiment described herein is to be applied mutatis mutandis to each and every embodiment unless specifically stated otherwise.

### 2. Compositions and Methods for Modulating Cytokine Production Vascular Leakage in Flavivirus Infections

The present invention is based in part on the determination that Flavivirus sNS1 is a pathogen-associated molecular pattern (PAMP) recognized by TLR4 and signals through this pattern recognition receptor to elicit production of pro-inflammatory cytokines such as TNF-α, IL-6, IFN-β, IL-1β, IL-8 and IL-12, IP-10 and MCP-1. These pro-inflammatory cytokines together with direct action of NS1 on endothelial cells, which also express TLR4 on their cell surface, results in an increase in vascular permeability. The striking parallels to LPS pathophysiology indicate that sNS1 is a viral counterpart of bacterial endotoxin and that TLR4 signaling is a target for inhibiting the development of Flavivirus-mediated disease, including the production of pro-inflammatory mediators and development of vascular leakage. Accordingly, the present invention provides methods and compositions that include a TLR4 antagonist for modulating the production pro-inflammatory mediators and/or vascular leakage in Flavivirus infections including in the treatment and prevention of Flavivirus infections.

2.1 NS1 is Conserved Across the Flavivirus Genus

The Flavivirus NS1 glycoprotein comprises about 350 amino acids and has a molecular weight of 48-55 kDa, depending on its glycosylation status. It exists in multiple oligomeric forms and is found in different cellular locations: a cell membrane-bound form in association with virus-induced intracellular vesicular compartments, on the cell surface and as a soluble secreted hexameric lipoparticle (i.e., sNS1). Because NS1 is structurally conserved across all members of the genus, the present inventors propose that Flavivirus sNS1 glycoproteins generally are recognized as PAMPs by TLR4 and that TLR4 antagonists are useful for treating or preventing disease associated with any Flavivirus species including but not limited to: Aroa virus, Bussuquara virus, Iguape virus, Naranjal virus, Dengue virus group, Dengue virus, Dengue virus 1, Dengue virus 2, Dengue virus 3, Dengue virus 4, Japanese encephalitis virus group, Japanese encephalitis virus, Japanese encephalitis virus strain JAOARS982, Japanese encephalitis virus strain Nakayama, Japanese encephalitis virus strain SA(V), Japanese encephalitis virus strain SA-14, Koutango virus, Murray Valley encephalitis virus, Alfuy virus, Murray valley encephalitis virus (strain MVE-1-51), St. Louis encephalitis virus, St. Louis encephalitis virus (strain MS1-7), Usutu virus, West Nile virus, Kurjin virus, West Nile virus crow/New York/3356/2000, West Nile virus H442, West Nile virus SA381/00, West Nile virus SA93/01, West Nile virus SPU116/89, West Nile virus strain 385-99, West Nile virus strain PT5.2, West Nile virus strain PT6.16, West Nile virus strain PT6.39, West Nile virus strain PT6.5, West Nile virus strain PTRoxo, Kokobera virus group, Kokobera virus, New Mapoon virus, Stratford virus, unclassified Kokobera virus group, CY1014 virus, Modoc virus group, Cowbone Ridge virus, Jutiapa virus, Modoc virus, Sal Vieja virus, San Perilta virus, mosquito-borne viruses, Ilheus virus, Rocio virus, Sepik virus, Ntaya virus group, Bagaza virus, Israel turkey meningoencephalomyelitis virus, Ntaya virus, Tembusu virus, Sitiawan virus, Yokose virus, Rio Bravo virus group, Apoi virus, Bukalasa bat virus, Carey Island virus, Dakar bat virus, Entebbe bat virus, Rio Bravo virus, Saboya virus, Potiskum virus, Seaborne tick-borne virus group, Mcaban virus, Saumarez Reef virus, Tyuleniy virus, Spondwenl virus group, Zika virus, Spondweni virus, tick-borne encephalitis virus group, Kyasanur forest disease virus, Alkhurma hemorrhagic fever virus, Langat virus, Langat virus (strain TP21), Langat virus (strain Yelantsev), Louping ill virus, Louping ill virus (strain 31), Louping ill virus (strain K), Louping ill virus (strain Negishi 3248/49/P10), Louping ill virus (strain Norway), Louping ill virus (strain SB 526), Omsk hemorrhagic fever virus, Phnom Penh bat virus, Powassan virus, Deer tick virus, Tick-borne powassan virus (strain 1b), Royal Farm virus, Karshi virus, Tick-borne encephalitis virus, Kumlinge virus, Negishi virus, Tick-borne encephalitis virus (strain HYPR), Tick-borne encephalitis virus (STRAIN SOFJIN), Tick-borne encephalitis virus (WESTERN SUBTYPE), Turkish sheep encephalitis virus, Yaounde virus, Yellow fever virus group, Banzi virus, Bouboul virus, Edge Hill virus, Uganda S virus, Wesselsbron virus, Yellow fever virus, Yellow fever virus 17D, Yellow fever virus 1899/81, Yellow fever virus isolate Angola/14FA/1971, Yellow fever virus isolate Ethiopia/Couma/1961, Yellow fever virus isolate Ivory Coast/1999, Yellow fever virus isolate Ivory Coast/85-82H/1982, Yellow fever virus isolate Uganda/A7094A4/1948, Yellow fever virus strain French neurotropic vaccine, Yellow fever virus strain Ghana/Asibi/1927, Yellow fever virus Trinidad/79A/1979, unclassified Flavivirus, Aedes flavivirus, Batu Cave virus, Cacipacore virus, Cell fusing agent virus, Chaoyang virus, Chimeric Tick-borne encephalitis virus/Dengue virus 4, Culex flavivirus, Flavivirus CbaAr4001, Flavivlrus FSME, Flavivirus SST-2008, Gadgets Gully virus, Greek goat encephalitis virus, Jugra virus, Kadam virus, Kamiti River virus, Kedougou virus, Montana myotis leukoencephalitis virus, Ngoye virus, Nounane virus, Quang Binh virus, Russian Spring-Summer encephalitis virus, Sokoluk virus, Spanish sheep encephalitis virus, T'Ho virus, Tai forest virus 831, Tamana bat virus, Tick-borne flavivirus, Wang Thong virus, and Flavivirus sp.

2.2 TLR4 Antagonists

The TLR4 antagonist includes and encompasses any active agent that inhibits signaling through the TLR4 pathway, reduces the accumulation, function or stability of TLR4; or decreases expression of the TLR4 gene, and such inhibitors include without limitation, small molecules and macromolecules such as nucleic adds, peptides, polypeptides, peptidomimetics, carbohydrates, saccharides, liposaccharides, polysaccharides, lipopolysaccharides, lipids or other organic (carbon containing) or inorganic molecules.

In some embodiments, the TLR4 antagonist is an antagonistic nucleic acid molecule that functions to inhibit the transcription or translation of TLR4 transcripts. Representative transcripts of this type include nucleotide sequences corresponding to any one the following sequences: (1) human TLR4 nucleotide sequences as set forth for example in GenBank Accession Nos. NM_138554, NM_003266 and NM_138557; (2) nucleotide sequences that share at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) nucleotide sequences that hybridize under at least low, medium or high stringency conditions to the sequences referred to in (1); (4) nucleotide sequences that encode any one of the following amino add sequences: human TLR4 amino acid sequences as set forth for example in GenPept Accession Nos. NP_612564.1, NP_003257.1 and NP_612567.1; (5) nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (4).

Illustrative antagonist nucleic add molecules include antisense molecules, aptamers, ribozymes and triplex forming molecules, RNAi and external guide sequences. The nucleic acid molecules can act as effectors, inhibitors, modulators, and stimulators of a specific activity possessed by a target molecule, or the functional nucleic acid molecules can possess a de novo activity independent of any other molecules.

Antagonist nucleic add molecules can interact with any macromolecule, such as DNA, RNA, polypeptides, or carbohydrate chains. Thus, antagonist nucleic add molecules can interact with TLR4 mRNA or the genomic DNA of TLR4 or they can interact with a TLR4 polypeptide. Often antagonist nucleic acid molecules are designed to interact with other nucleic acids based on sequence homology between the target molecule and the antagonist nucleic add molecule. In other situations, the specific recognition between the antagonist nucleic acid molecule and the target molecule is not based on sequence homology between the antagonist nucleic acid molecule and the target molecule, but rather is based on the formation of tertiary structure that allows specific recognition to take place.

In some embodiments, anti-sense RNA or DNA molecules are used to directly block the translation of TLR4 by binding to targeted mRNA and preventing protein translation. Antisense molecules are designed to interact with a target nucleic acid molecule through either canonical or non-canonical base pairing. The interaction of the antisense molecule and the target molecule may be designed to promote the destruction of the target molecule through, for example, RNAseH mediated RNA-DNA hybrid degradation. Alternatively the antisense molecule may be designed to interrupt a processing function that normally would take place on the target molecule, such as transcription. Antisense molecules can be designed based on the sequence of the target molecule. Numerous methods for optimization of antisense efficiency by finding the most accessible regions of the target molecule exist. Non-limiting methods include in vitro selection experiments and DNA modification studies using DMS and DEPC. In specific examples, the antisense molecules bind the target molecule with a dissociation constant (K_{d}) less than or equal to 10⁻⁶, 10⁻⁸, 10⁻¹⁰, or 10⁻¹². In specific embodiments, antisense oligodeoxyribonucleotides derived from the translation initiation site, e.g., between −10 and +10 regions are employed. Illustrative TLR4 antisense compounds are disclosed for example in U.S. Pat. App. Pub. No. 2010/0111936, which is incorporated herein by reference in its entirety.

In other embodiments, RNA molecules that mediate RNA interference (RNAi) of the TLR4 gene or transcript thereof can be used to reduce or abrogate gene expression. RNAi refers to interference with or destruction of the product of a target gene by introducing a single-stranded or usually a double-stranded RNA (dsRNA) that is homologous to the transcript of a target gene. RNAi methods, including double-stranded RNA interference (dsRNAi) or small interfering RNA (siRNA), have been extensively documented in a number of organisms (Fire et al., 1998. Nature 391, 806-811). In mammalian cells, RNAi can be triggered by 21- to 23-nucleotide (nt) duplexes of small interfering RNA (siRNA) (Chiu et al., 2002 Mol. Cell. 10:549-561; Elbashir et al., 2001. Nature 411:494-498), or by micro-RNAs (miRNA), functional small-hairpin RNA (shRNA), or other dsRNAs which are expressed in vivo using DNA templates with RNA polymerase III promoters (Zeng et al., 2002. Mol. Cell 9:1327-1333; Paddison et al., 2002. Genes Dev. 16:948-958; Lee et al., 2002. Nature Biotechnol. 20:500-505; Paul et al., 2002. Nature Biotechnol. 20:505-508; Tuschl, T., 2002. Nature Biotechnol. 20:440-448; Yu et al., 2002. Proc. Natl. Acad. Sci. USA 99(9):6047-6052; McManus et al., 2002. RNA 8:842-850; Sui et al., 2002. Proc. Natl. Acad. Sci. USA 99(6):5515-5520).

In specific embodiments, dsRNA per se and especially dsRNA-producing constructs corresponding to at least a portion of the TLR4 gene are used to reduce or abrogate its expression. RNAi-mediated inhibition of gene expression may be accomplished using any of the techniques reported in the art, for instance by introducing a nucleic acid construct encoding a stem-loop or hairpin RNA structure into the genome of the target cell, or by expressing a transfected nucleic acid construct having homology for a TLR4 gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts, which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are suitably at least about 100 nucleotides in length. RNA-encoding constructs that express a single RNA designed to form a dsRNA via internal folding are usually at least about 200 nucleotides in length.

The promoter used to express the dsRNA-forming construct may be any type of promoter, provided it is operable in the cell in which a target transcript is expressed.

In some embodiments, RNA molecules of about 21 to about 23 nucleotides, which direct cleavage of specific mRNA to which they correspond, as for example described by Tuschl et al. in U.S. Pat. App. Pub. No. 2002/0086356, can be utilized for mediating RNAi. Such 21- to 23-nt RNA molecules can comprise a 3′ hydroxyl group, can be single-stranded or double stranded (as two 21- to 23-nt RNAs) wherein the dsRNA molecules can be blunt ended or comprise overhanging ends (e.g., 5′, 3′).

In some embodiments, the antagonist nucleic acid molecule is a siRNA. siRNAs can be prepared by any suitable method. For example, reference may be made to International Publication WO 02/44321, which discloses siRNAs capable of sequence-specific degradation of target mRNAs when base-paired with 3′ overhanging ends, which is incorporated by reference herein. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER™ siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. In addition, methods for formulation and delivery of siRNAs to a subject are also well known in the art. See, e.g., U.S. Pat. App. Pub. Nos. 2005/0282188; 2005/0239731; 2005/0234232; 2005/0176018; 2005/0059817; 2005/0020525; 2004/0192626; 2003/0073640; 2002/0150936; 2002/0142980; and 2002/0120129, each of which is incorporated herein by reference.

Illustrative RNAi molecules (e.g., TLR4 siRNA or shRNA) are described in the art (e.g., Jiang, et al., 2011. Am J Transplant. 11(9):1835-1844; U et al., 2011. Appl Microbiol Biotechnol. 92(1):115-124; Yang et al., 2010. J Exp Clin Cancer Res. 29:92; Hua et al., 2009. Mol Immunol. 46(15):2876-2884; and Wu et al., 2012. J Biomed Biotechnol. 2012:406435) or available commercially from Santa Cruz Biotechnology, Inc. (Santa Cruz, Calif., USA), GeneCopoiea (Rockville, Md., USA) and OriGene Technologies, Inc. (Rockville, Md., USA).

In another aspect, the TLR-4 antagonist can be a nucleic acid aptamer (DNA or RNA). Aptamers can be selected from libraries screened for their ability to bind TLR-4 and perturb its activity. The techniques for selecting aptamers against specific targets, forming multivalent aptamers based upon the selected individual aptamers, and their use have been described. See, e.g., U.S. Pat. No. 6,458,559 to Shi et al., and U.S. Pat. App. Pub. No. 2004/0053310 to Shi et al., each of which is hereby incorporated by reference in its entirety.

The present invention also contemplates carbohydrate agents that antagonize TLR4 signaling. Such molecules include for example liposaccharide compounds including lipid A analogs represented by formula (I):

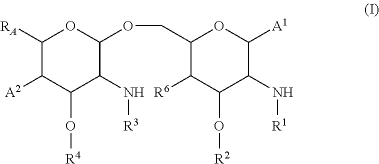

where R¹ is selected from the group consisting of:

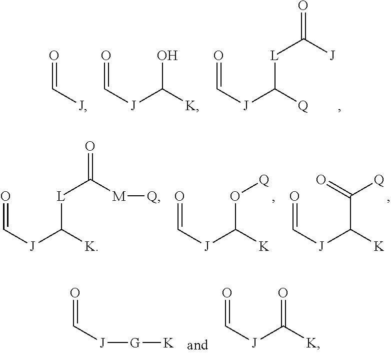

where each J, K, and Q, Independently, is straight or branched C₁ to C₁₅ alkyl; L is O, NH, or CH₂; M is O or NH; and G is NH, O, S, SO, or SO₂,

R² is straight or branched C₅ to C₁₅ alkyl;

R³ is selected from the group consisting of straight or branched C₅ to C₁₈ alkyl,

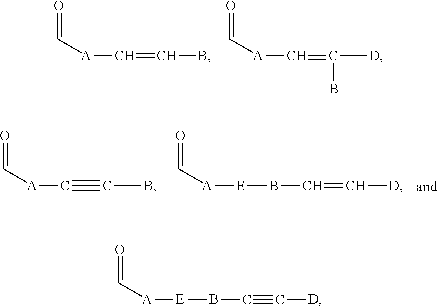

where E is NH, O, S, SO, or SO₂; each A, B, and D, independently, is straight or branched C₁ to C₁₅ alkyl;

R⁴ is selected from the group consisting of straight or branched C₄ to C₂₀ alkyl, and

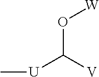

where each U and V, independently, is straight or branched C₂ to C₁₅ alkyl and W is hydrogen or straight or branched C₁ to C₅ alkyl;

R_{A} is R⁵ or R⁵—O—CH₂—, R⁵ being selected from the group consisting of hydrogen, J′, -J′-OH, -J′-O—K′, -J′-O—K′—OH, and -J′-O—PO(OH)₂, where each J′ and K′, independently, is straight or branched C₁ to C₅ alkyl;

R⁶ is selected from the group consisting of hydroxy, halogen, C₁ to C₅ alkoxy, and C₁ to C₅ acyloxy;

A¹ and A², independently, are selected from the group consisting of OH,

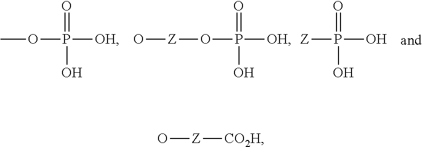

where Z is straight or branched C₁ to C₁₀ allyl;

or a pharmaceutically acceptable salt or phosphate ester thereof.

In specific embodiments, the liposaccharide compound of formula (I) is represented by formula (II):

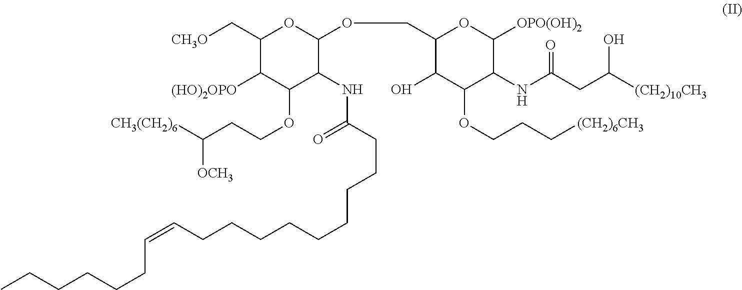

or a pharmaceutically acceptable salt or phosphate ester thereof.

In some embodiments, the lipopolysaccharide compound of formula (I) is represented by formula (III):

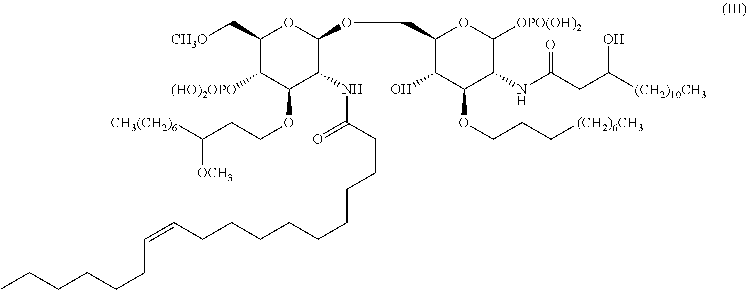

or a pharmaceutically acceptable salt or phosphate ester thereof.

Compounds falling within the scope of formula (I) Inhibit dimerization of TLR4, thereby blocking its activation. In an illustrative example, the liposaccharide compound of formula (I) is eritoran tetrasodium (also known as compound E5664). Eritoran tetrasodium is the tetrasodium salt of the compound shown immediately above. Eritoran tetrasodium is described in U.S. Pat. No. 5,935,938, which is incorporated by reference herein in its entirety.

Other illustrative liposaccharide compounds for antagonizing TLR4 include the following compounds:

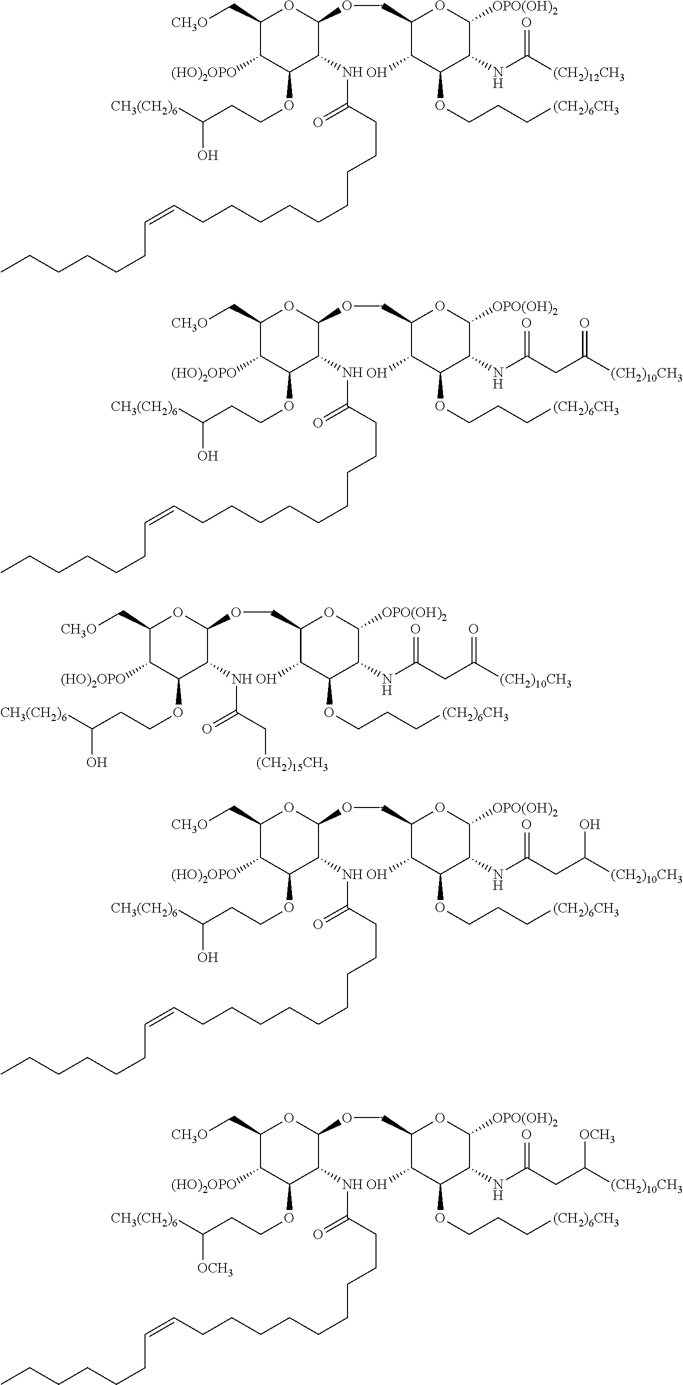

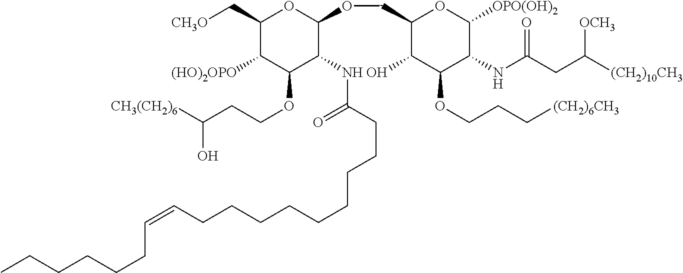

or pharmaceutically acceptable salts thereof or phosphate ester thereof, as described for example in U.S. Pat. App. Pub. No. 2007/0072824, which is incorporated by reference herein in its entirety.

Alternative liposaccharide TLR4 antagonists that can be used in the invention include, for example, compound B531 (U.S. Pat. No. 5,530,113), as well as other compounds described in the following patents: U.S. Pat. No. 5,935,389 (e.g., substituted liposaccharides identified by formula (I)); U.S. Pat. No. 5,612,476 (e.g., lipid A analogs disclosed at columns 2-41); U.S. Pat. No. 5,756,718 (lipid A analogs disclosed at columns 2-40); U.S. Pat. No. 5,843,918 (e.g., lipid A analogs disclosed at columns 2-48); U.S. Pat. No. 5,750,664 (e.g., substituted liposaccharides identified by formula I); U.S. Pat. No. 6,235,724 (e.g., lipid A analogs identified by formulas (I) and (II)); U.S. Pat. No. 6,184,366 (e.g., lipid A analogs identified by formula (I)), U.S. Pat. No. 5,681,824, and U.S. Pat. App. Pub. Nos. 2003/0144503 and 2002/0028927. Methods for making these compounds are also described within these documents. Additional methods for making such compounds are described, for example, in International Publication WO 02/94019. Each of the patent documents referred to above is incorporated by reference herein in its entirety.

In other embodiments, TLR4 antagonist compounds are selected from lipid compounds, non-limiting examples of which are represented by formula (IV):

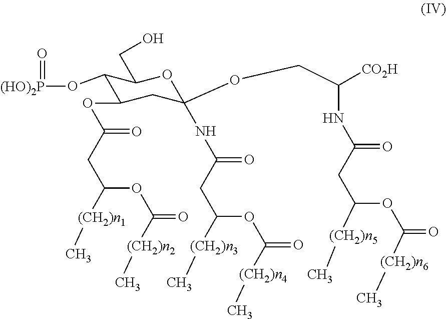

or a pharmaceutically acceptable salt or phosphate ester thereof; wherein n₁, n₃, and n₅, are the same or different and are positive integers from, for example, 1 to about 10 (e.g., 10); n₂, n₄, and n₆, are the same or different and are positive integers less than 8. Compounds of formula (III) are synthetic lipid A mimetics that do not stimulate cytokine production or other gene expression in human peripheral blood monocytes in vitro or induce an inflammatory response in vivo, as described for example by Stöver et al. (2004. J. Biol Chem. 279(6):4440-4449, incorporated herein by reference).

In non-limiting examples, at least one of n₂, n₄, and n₆ is less than 7 so that at least one secondary acyl group of formula (IV) is less than 10 carbons. Compounds of formula (IV) with at least one secondary acyl group less than 10 carbons have been shown to be potent TLR4 antagonists (see, Stöver et al., 2004, supra).

In specific embodiments, the TLR4 antagonist of formula (IV) has the following structure:

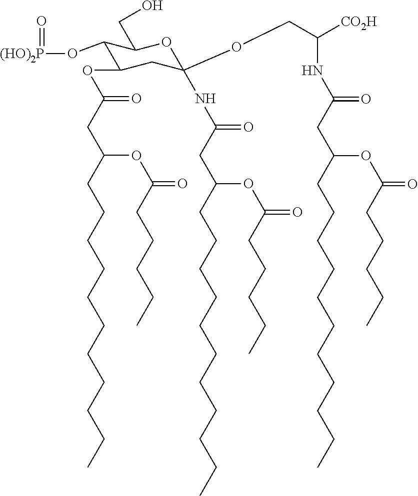

or a pharmaceutically acceptable salt or phosphate ester thereof. The above-identified TLR4 antagonist is commercially available from GlaxoSmithKline (UK) under the trade name CRX 526, as described for example by Fort et al. (2005. Journal of Immunology 174: 6416-6423, incorporated herein by reference).

In other embodiments, the TLR4 antagonist of formula (IV) is a compound selected from one of the following:

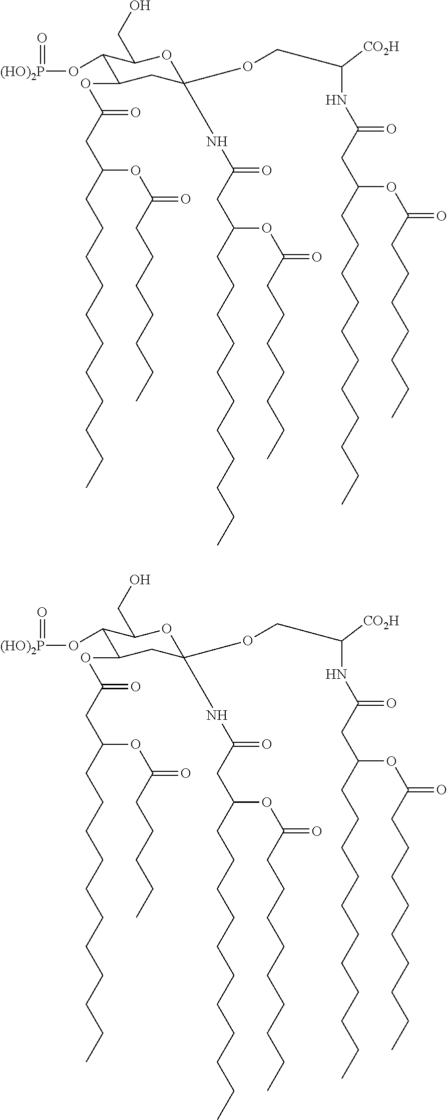

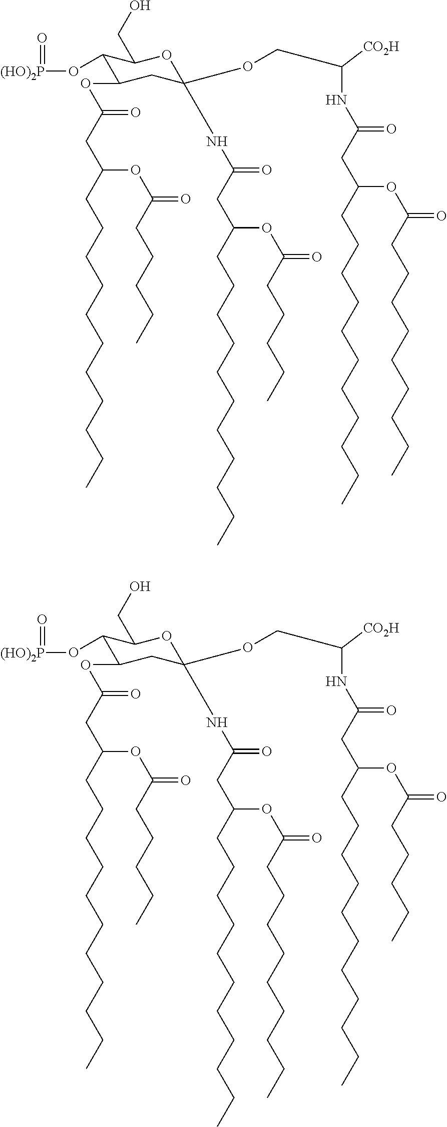

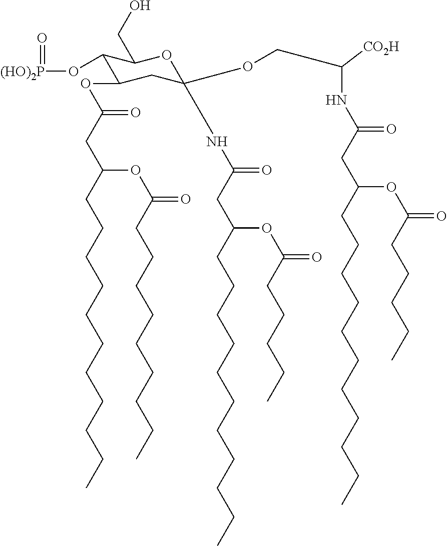

or pharmaceutically acceptable salts and phosphate esters thereof. The above identified examples of formula (III) are identified by Stöver et al. (2004, supra) as being synthetic lipid A mimetics and were synthesized as described in Johnson et al. (1999, Bioorg Med Chem. Lett. 9(15):2273-2278, incorporated herein by reference).

In specific embodiments, the small molecule TLR4 antagonist is a compound of formula (I):

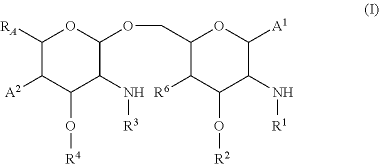

where R¹ is selected from the group consisting of:

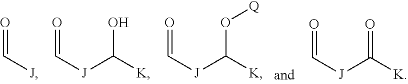

where each J, K, and Q, independently, is straight or branched C₁ to C₁₅ alkyl;

R² is straight or branched C₅ to C₁₅ alkyl;

R³ is selected from the group consisting of straight or branched C₅ to C₁₈ alkyl and

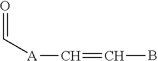

where A and B are each independently straight or branched C₁ to C₁₅ alkyl;

R⁴ is selected from the group consisting of straight or branched C₄ to C₂₀ alkyl, and

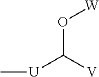

where each U and V, independently, is straight or branched C₂ to C₁₅ alkyl and W is hydrogen or straight or branched C₁ to C₅ alkyl;

R_{A} is R⁵ or R⁵—O—CH₂—, R⁵ being selected from the group consisting of hydrogen, J′, -J′-OH, -J′-O—K′, -J′-O—K′—OH, and -J′-O—PO(OH)₂, where each J′ and K′, independently, is straight or branched C₁ to C₅ alkyl;

R⁶ is selected from the group consisting of hydroxy, halogen, C₁ to C₅ alkoxy, and C₁ to C₅ acyloxy;

A¹ and A² are each independently

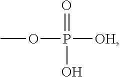

or a pharmaceutically acceptable salt or phosphate ester thereof.

In some embodiments, R¹ is

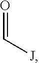

where J is straight or branched C₁₀ to C₁₅ alkyl.

In other embodiments, R¹ is

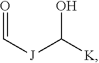

where J is straight or branched C₁ to C₃ alkyl and K is straight or branched C₈ to C₁₅ alkyl.

In still other embodiments, R¹ is

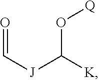

where J is straight or branched C₁ to C₃ alkyl, K is straight or branched C₈ to C₁₅ alkyl and Q is straight or branched C₁ to C₃ alkyl.

In further embodiments, R¹ is

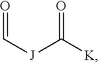

where J is straight or branched C1 to C3 alkyl and K is straight or branched C8 to C15 alkyl.

For example, in some embodiments, R¹ is

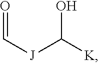

where J is —CH₂— and K is straight or branched C₁₀ to C₁₃ alkyl.

In other embodiments, R¹ is

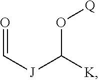

where J is —CH₂—, K is straight or branched C₁₀ to C₁₃ alkyl and Q is straight or branched —CH₃.

In further embodiments, R¹ is

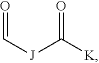

where J is —CH₂— and K is straight or branched C₁₀ to C₁₃ alkyl.

In some embodiments, R² is straight or branched Ca to C₁₂ alkyl, e.g., straight or branched C₁₀ alkyl.

In some embodiments, R³ is straight or branched C₁₀ to C₁₈ acyl, e.g., C₁₈ acyl.

In other embodiments, R³ is

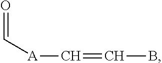

where A is straight or branched C₇ to C₁₂ alkyl and B is straight or branched C₄ to C₉ alkyl.

For example, in some embodiments, R³ is

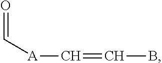

where A is straight or branched C₉ alkyl and B is straight or branched C₆ alkyl.

In some embodiments, R₄ is straight or branched C₈ to C₁₂ alkyl, e.g., straight or branched C₁₀ alkyl.

In other embodiments, R⁴ is

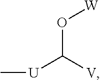

where U is straight or branched C₂ to C₄ alkyl, V is straight or branched C₅ to C₉ alkyl and W is hydrogen or —CH₃.

For example, in some embodiments, R⁴ is

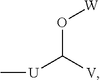

where U is straight or branched C₂ alkyl, V is straight or branched C₇ alkyl and W is hydrogen or —CH₃.

In some embodiments, R_{A} is R⁵ or R⁵—O—CH₂—, where R⁵ is J′ and where J is straight or branched C₁ to C₅ alkyl.

In some embodiments, R_{A} is R₅—O—CH₂—, where R⁵ is —CH₃.

In some embodiments, R⁶ is hydroxyl.

In further embodiments, the TLR4 antagonist is a compound of formula (I):

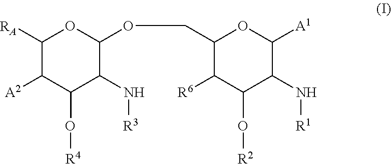

where R¹ is selected from the group consisting of:

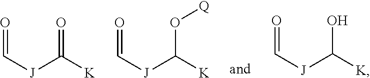

where J is straight or branched C₁ to C₃ alkyl, K is straight or branched C₈ to C₁₅ alkyl and Q is straight or branched C₁ to C₃ alkyl;

R² is straight or branched C₈ to C₁₂ alkyl;

R³ is

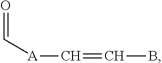

where A is straight or branched C₇ to C₁₂ alkyl and B is straight or branched C₄ to C₉ alkyl;

R⁴ is selected from straight or branched C₈ to C₁₂ alkyl and

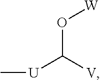

where U is straight or branched C₂ to C₄ alkyl, V is straight or branched C₅ to C₉ alkyl and W is hydrogen or —CH.sub.₃; R_{A} is R⁵—O—CH₂—, where R⁵ is J′ and where J′ is straight or branched C₁ to C₅ alkyl; R.sup.6 is hydroxyl; A¹ and A² are each independently

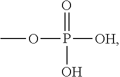

or pharmaceutically acceptable salt or phosphate ester thereof.

In still further embodiments, the TLR4 antagonist is a compound of formula (I):

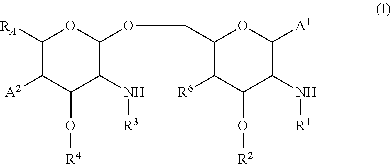

where R¹ is selected from the group consisting of:

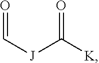

where J is straight or branched C₁ to C₃ alkyl, K is straight or branched Ca to Cis alkyl and Q is straight or branched C₁ to C₃ alkyl;

R² is straight or branched C₈ to C₁₂ alkyl;

R³ is

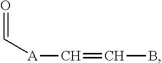

where A is straight or branched C₇ to C₁₂ alkyl and B is straight or branched C₄ to C₉ alkyl

R⁴ is

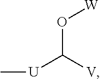

where U is straight or branched C₂ to C₄ alkyl, V is straight or branched C₅ to C₉ alkyl and W is hydrogen or —CH₃; R_{A} is R⁵—O—CH₂—, where R⁵ is J′ and where J′ is straight or branched C₁ to C₅ alkyl; R⁶ is hydroxyl; A¹ and A² are each independently

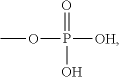

or a pharmaceutically acceptable salt or phosphate ester thereof.

Compounds according to any one of formulas (I), (II) (III) and (IV) may be prepared in the form of a micelle, as described in U.S. Pat. No. 6,906,042, which is incorporated herein by reference in its entirety for the description of such micelles and methods for preparing same.

In other embodiments, the TLR4 antagonist is an antagonist MD-2 or TLR4 polypeptide, such as a polypeptide fragment of MD-2 or TLR4 that corresponds to at least a portion of a polypeptide of the MD-2/TLR4 receptor complex and binds TLR4 ligand during TLR4 signal transduction. Other examples of TLR4 antagonists include a non-TLR4 protein or polypeptide that inhibits TLR4 activity, a small molecule inhibitor of TLR4 activity, or an inhibitory ligand that is a variant of the natural ligand of TLR4, namely bacterial lipopolysaccharide (e.g., analogs of the lipid A region of LPS as described above). Regardless of the type of TLR4 antagonist employed, the TLR4 antagonist can be administered to achieve at least transient blockade of TLR4 function, thereby neutralizing or at least partially inhibiting the effect of TLR4 on Flavivirus mediated disease.

In representative examples, antagonist polypeptide fragments of TLR4, which can function as TLR4 decoy polypeptides, include short polypeptides from about 10 to 100 or 10 to 50 amino acids in length (or smaller), which contain a TLR-4 ligand binding domain. These peptide fragments can also be part of an N-terminal or C-terminal fusion protein. The full-length sequence of various human TLR4 isoforms are known (see GenBank Accession Nos. NP_612564.1 (isoform A), NP_003257.1 (isoform C), and NP_612567.1 (isoform D), each of which is hereby incorporated by reference in its entirety). Sequences for other mammalian TLR-4 homologs are also known, including those of mouse, rat, orangutan, etc. Non-limiting examples of TLR4 decoy polypeptides are disclosed in U.S. Pat. App. Pub. Nos. 2013/0203649, 2005/0112659 and 2006/0241040, which are incorporated herein by reference in their entirety.

Non-TLR4 protein or polypeptide inhibitors of TLR4 have also been identified in the literature, and these can be used in the methods and compositions of the present invention. Two such inhibitors are identified in Yang et al., Novel TLR4 Antagonizing Peptides Inhibit LPS-Induced Release of Inflammatory Mediators by Monocytes, Biochem. Biophys. Res. Commun. 329(3): 846-54 (2005); and chemokine receptor 4 and its ligand have also been shown to be effective (Kishore et al., Selective Suppression of Toll-like Receptor 4 Activation by Chemokine Receptor 4, FEBS Lett. 579(3): 699-704 (2005)). Short-form human md-2 polypeptides are disclosed by Ariditi et al. in U.S. Pat. App. Pub. Nos. 2014/0045775, which is incorporated herein by reference in its entirety, as negative regulators of TLR4 signaling. Peptide fragments of surfactant protein-A and derivatives thereof are also known to block TLR4 signaling as disclosed for example in U.S. Pat. App. Pub. Nos. 2014/0256613, which is incorporated herein by reference in its entirety.

Another example of a TLR4 antagonist that can be used in methods of the present invention is Rhodobacter sphaeroides lipid A (RSLA). RSLA has five acyl chains compared with six chains on Lipid A from most Gram negative bacteria, has pronounced antagonistic activity for other Gram negative Lipid A, and only minor agonist activity on some cell types (Kutuzova et al., 2001. J. Immunol. 167:482-489; Golenbock et al., 1991. J. Biol Chem. 266:19490-19498; Qureshi et al., 1991, Infection and Immunity 59:441-444), which are incorporated herein by reference in their entirety.

The present invention also contemplates small molecule TLR4 antagonists. Non-limiting small molecule compounds are disclosed for example in U.S. Pat. App. Pub. No. 2013/028139 to Wipf et al., which is incorporated herein by reference in its entirety. Specific embodiments of the compounds disclosed by Wipf et al. include:

(1) 4-O-(3-O-{2-(acetylamino)-2-deoxy-4-O-(6-deoxyhexopyranosyl)-3-O-[2-O-(6-deoxyhexopyranosyl)hexopyranosyl]hexopyranosyl}hexopyranosyl) hexopyranose (Compound 3), having the structure:

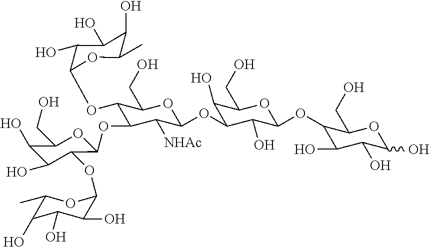 [SMILES: CC(=O)NC1C(OC2C(O)C(CO)OC(OC3C(CO)OC(O)C(O)C3O)C2O)OC(CO)C(OC2OC(C)C(O)C(O)C2O)C1OC1OC(CO)C(O)C(O)C1OC1OC(C)C(O)C(O)C1O]

(2) 3-acetamido-4,5-dihydroxy-6-(hydroxymethyl)tetrahydro-2H-pyran-2-yl dihydrogen phosphate, sodium salt (Compound 4), having the structure:

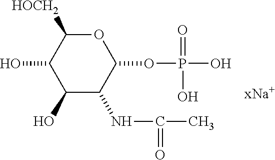

(3) cyclohexanamine compound with 1,6-di-O-phosphono-beta-D-glycero-hexopyranose (4:1) hydrate (Compound 8), having the structure:

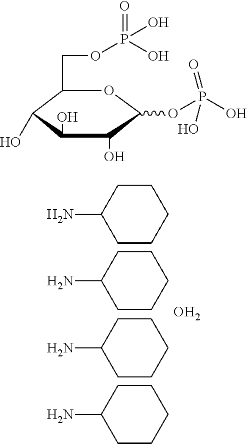

(4) 2-(acetylamino)-2-deoxy-D-galactopyranose hydrate (Compound 16), having the structure:

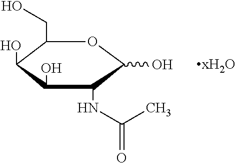

(5) 2-(acetylamino)-2-deoxy-4-O-hexopyranosylhexopyranose (Compound 27), having the structure:

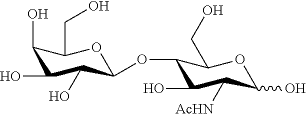

(6) isopropyl 3,4,6-tri-O-acetyl-2-(acetylamino)-2-deoxyhexopyranoside (Compound 34), having the structure:

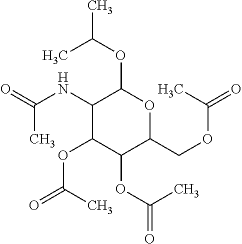 [SMILES: CC(=O)NC1C(OC(C)C)OC(COC(C)=O)C(OC(C)=O)C1OC(C)=O]

(7) derivatives of isopropyl 3,4,6-tri-O-acetyl-2-(acetylamino)-2-deoxyhexopyranoside (Compound 34), represented by formula (V):

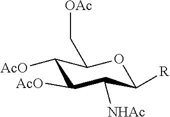

where R is selected from R¹ and —O—R¹, where R¹ may be a substituted or unsubstituted alkane or alkene, where the substituent if present may be methyl or ethyl, where the alkane or alkene portion optionally comprises a branched or cyclic component, and may have between 1 and 12 or between 1 and 6 carbon atoms.

(8) derivatives of isopropyl 3,4,6-tri-O-acetyl-2-(acetylamino)-2-deoxyhexopyranoside (Compound 34), represented by formula (VI):

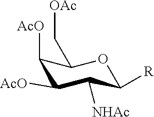

where R is selected from R¹ and —O—R¹, where R¹ may be a substituted or unsubstituted alkane or alkene, where the substituent if present may be methyl or ethyl, where the alkane or alkene portion optionally comprises a branched or cyclic component, and may have between 1 and 12 or between 1 and 6 carbon atoms.

In specific, non-limiting embodiments, a derivative of isopropyl 3,4,6-tri-O-acetyl-2-(acetylamino)-2-deoxyhexopyranoside may be selected from the following group of compounds:

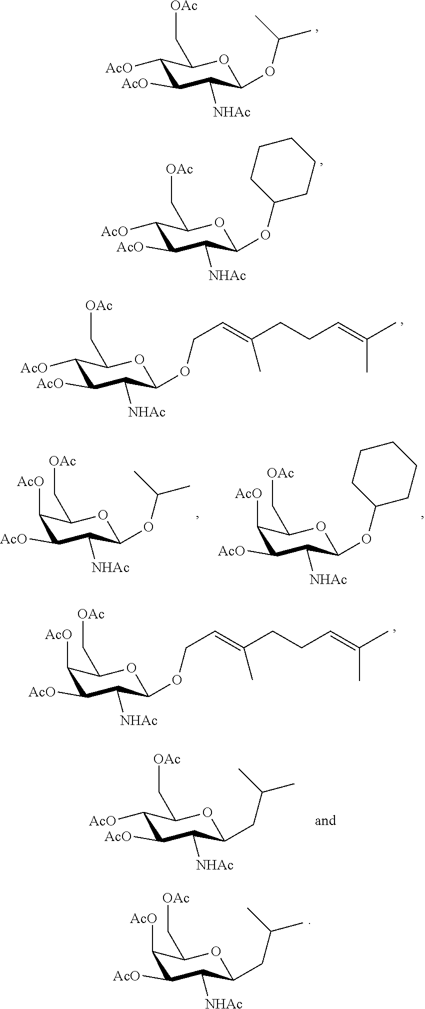

In illustrative examples, small molecule compounds disclosed by Wipf et al. are selected from compounds 1, 3, 4, 5, 6, 8, 16, 21, 22, 27, 28, 29, 30, 45, and 47 listed in Table 1 of Wipf et alt.

Other examples of TLR4 antagonists include, without limitation TAK-242 (see, Ii et al., A Novel Cyclohexene Derivative, (TAK-242), Selectively Inhibits Toll-like Receptor 4-mediated Cytokine Production Through Suppression of Intracellular Signaling, Mol. Pharmacol. 69(4): 128 8-95 (2006)); the endogenous TLR4 Inhibitor RP1O5 (see, Divanovic et al., Inhibition of TLR4/MD-2 signaling by RP1O5/MD-1, J. Endotoxin Res. 11(6): 363-368 (2005)); CyP, a natural LPS mimetic derived from the cyanobacterilum Oscillatoria planktothrix FP1 (see, Macagno et al., A Cyanobacterial LPS Antagonist Prevents Endotoxin Shock and Blocks Sustained TLR4 Stimulation Required for Cytokine Expression, J Exp Med. 203(6): 1481-1492 (2006)); a phenol/water extract from T. socranskii subsp. socranskii (TSS-P) (see, Lee et al., Phenol/water Extract of Treponema socranskii subsp. socranskii as an Antagonist of Toll-like Receptor 4 Signaling, Microbiol. 152(2): 535-46 (2006)); CLR proteins such as Monarch-i (see, Williams et al., The CATERPILLER Protein Monarch-i is an Antagonist of Toll-like Receptor-, Tumor Necrosis Factor alpha-, and Mycobacterium tuberculosis-induced pro-inflammatory signals, J Biol Chem. 280(48): 39914-39924 (2005)); and small molecule TLR-4/TLR-2 dual antagonists, such as ER811243, ER811211, and ER811232 (see, U.S. Patent App. Pub. No. 2005/0113345 to Chow et al.). Further examples of TLR4 inhibitors or antagonists can be found in International Publication WO2006/138681A2. The above disclosures are hereby incorporated by reference herein in their entirety.

The present invention also contemplates anti-TLR4 antagonist antibodies. Numerous antagonist antibodies are disclosed in the art, illustrative examples of which include as described for example in U.S. Pat. App. Pub. No. 2009/0136509 to Blake (e.g., antibodies based on monoclonal antibody MTS510) and U.S. Pat. App. Pub. No. 2012/0177648 to Kosco-Vilbols et al., are hereby incorporated by reference herein in their entirety. Other TLR4 antagonist antibodies are available commercially, a non-limiting example of which is NI-0101, which is available from NovImmune SA (Plan-les-Ouates, Switzerland).

2.3 Screening Methods

The present invention also provides methods for the identification of agents suitable for use in the treatment or prevention of Flavivirus infection or symptom thereof. Antagonists identified by this method may be antagonists of TLR4 having any of the characteristics or effects described above. Antagonists identified by the methods described herein may be suitable for use in the treatment or prevention of Flavivirus infection, or in the treatment or prevention of any of the conditions or symptoms described herein, such as acute febrile illness, malaise, headache, flushing, diarrhea, nausea, vomiting, abdominal pain, myalgias and, in severe disease, production of pro-inflammatory mediators, including pro-inflammatory cytokines and vascular leakage.

Accordingly, the invention provides methods of identifying an agent for use in the treatment or prevention of a Flavivirus infection or symptom thereof. These methods generally comprise determining whether a test agent is capable of antagonizing TLR4. For example, the methods may involve determining whether a test agent is capable of decreasing the amount or activity of TLR4, wherein the ability to decrease the amount or activity of TLR4 indicates that the compound may be suitable for use in treating or preventing Flavivirus infection or symptom thereof as described herein. In some embodiments, the test agent is contacted with TLR4 or a nucleic acid sequenced sequence from which TLR4 is expressed.

A test agent for use in a screening method of the invention refers to any compound, molecule or agent that may potentially antagonize TLR4. The test agent may be, or may comprise, for example, a peptide, polypeptide, protein, antibody, polynucleotide, small molecule or other compound that may be designed through rational drug design starting from known antagonists of TLR4.

The test agent may be any agent having one or more characteristics of an antagonist of TLR4 as described above.

The test agent to be screened could be derived or synthesized from chemical compositions or man-made compounds. Candidate agents may be obtained from a wide variety of sources including libraries of synthetic or natural compounds. Suitable test agents which can be tested in the above assays include compounds derived from combinatorial libraries, small molecule libraries and natural product libraries, such as display (e.g., phage display) libraries. Multiple test agents may be screened using a method of the invention in order to identify one or more agents having a suitable effect on TLR4, such as inhibition of TLR4 activity or expression.

The screening methods of the invention may be carried out in vivo, ex vivo or in vitro. In particular, the step of contacting a test agent with TLR4 or with a cell or tissue that comprises TLR4 may be carried out in vivo, ex vivo or in vitro. The screening methods of the invention may be carried out in a cell-based or a cell-free system. For example, the screening method of the invention may comprise a step of contacting a cell or tissue comprising TLR4 with a test agent and determining whether the presence of the test agent leads to a decrease in the amount or activity of TLR4 in the cell or tissue.

For example, the ability of a test agent to decrease the activity or expression of TLR4 may be tested in a host cell or tissue that expresses TLR4. For example, the amount or activity of TLR4 may be assessed in vivo, ex vivo or in vitro in any suitable cells or tissue that express TLR4 (e.g., immune cells, endothelial cells, etc.).

In such a cell-based assay, the TLR4 and/or the test agent may be endogenous to the host cell or tissue, may be introduced into a host cell or tissue, may be introduced into the host cell or tissue by causing or allowing the expression of an expression construct or vector or may be introduced into the host cell or tissue by stimulating or activating expression from an endogenous gene in the cell.

In such a cell-based method, the amount of TLR4 may be assessed in the presence or absence of a test agent in order to determine whether the agent is altering the amount of TLR4 in the cell or tissue, such as through regulation of TLR4 expression in the cell or tissue or through destabilization of TLR4 protein within the cell or tissue. The presence of a lower TLR4 activity or a decreased amount of TLR4 within the cell or tissue in the presence of the test agent indicates that the test agent may be a suitable antagonist of TLR4 for use in accordance with the present invention in the treatment of an individual with a Flavivirus Infection or symptom thereof.

In one embodiment, such a cell based assay may be carried out in vitro or ex vivo on cells or tissue deriving from the patient to be treated. It may therefore be determined whether or not the test agent is capable of decreasing the activity or amount of TLR4 in the cells or tissue of that subject.

A method of the invention may use a cell-free assay. For example, the TLR4 may be present in a cell-free environment. A suitable cell-free assay may be carried out in a cell extract. For example, the contacting steps of the methods of the invention may be carried out in extracts obtained from cells that may express, produce or otherwise contain TLR4 and/or a test agent. A cell-free system comprising TLR4 may be incubated with the other components of the methods of the invention such a test agent.

In such a cell-free method, the amount of TLR4 may be assessed in the presence or absence of a test agent in order to determine whether the agent is altering the amount of TLR4 in the cell or tissue, such as through destabilization of TLR4 protein. In either case, the presence of a lower TLR4 activity or a decreased amount of TLR4 in the presence of the test agent indicates that the test agent may be a suitable antagonist of TLR4 for use in accordance with the present invention in the treatment of an individual with a Flavivirus infection or symptom thereof.

The contacting step(s) of the method of the invention may comprise incubation of the various components. Such incubations may be performed at any suitable temperature, typically between 4° C. and 40° C. Incubation periods may be selected for optimum activity, but may also be optimized to facilitate rapid high-throughput screening. Following the contact and optional incubation steps, the subject methods may further include a washing step to remove unbound components, where such a washing step is generally employed when required to remove label that would give rise to a background signal during detection, such as radioactive or fluorescently labeled non-specifically bound components.

Incubation in cell or cell-free assay systems may be performed in a microtiter plate (e.g., a 96-well plate or other microwell plate). Further, incubation may be performed in an automated fashion (e.g., for high-throughput screening).

A screening method of the invention may be carried out in vivo. For example, a screening method may be carried out in an animal model. In such an in vivo model, the effects of a test agent may be assessed in the circulation (e.g., blood), or in other organs such as the liver, kidney or heart. Suitably, the animal is a non-human animal such as a mouse or rat. Such a model may be used to assess the in vivo effects of a test agent. For example, such a model may be used to assess whether the test agent is capable of decreasing the activity or amount of TLR4 in vivo. In such a method, the amount of TLR4 may be assessed and/or the activity of TLR4 may be assessed.

An in vivo model may also be used to determine whether the test agent has any unwanted side effects. For example, a method of the invention may compare the effects of a test agent on TLR4 with its effects on other receptors in order to determine whether the test agent is specific.

In an in vivo model as described herein, or an in vitro model such as a cell-based or cell-free assay model as described herein, the effects of a test agent on TLR4 may be compared with the effects of the same agent on other Toll like receptors. As discussed above, a desirable TLR4 antagonist for use in method of treatment and prophylaxis as described herein may be an agent that selectively antagonizes TLR4. The screening methods of the invention may thus include an additional step of assessing whether the test agent has any effect on the activity or amount of one or more other Toll like receptors such as one or more Toll like receptors that are not TLR4. In such a method, a test agent may be identified as a suitable TLR4 antagonist if it is found to decrease the activity or amount of TLR4, but not to decrease, not to significantly decrease, not to significantly decrease, not to alter, or not to significantly alter, the activity or amount of one or more other Toll like receptors in the same assay.

In the screening methods described herein, the presence of a lower TLR4 activity or a decreased amount of TLR4 in the presence of the test agent indicates that the test agent may be a suitable antagonist of TLR4 for use in accordance with the present invention to treat an individual with a Flavivirus infection or symptom thereof.

A test agent that is an antagonist of TLR4 may result in a decrease in TLR4 activity or levels of at least 5%, at least 10%, at least 25%, at least 50%, at least 60%, at least 75%, or at least 85% or more in the presence of the test agent compared to in the absence of the test agent. A test agent that is an antagonist of TLR4 may result in a decrease in TLR4 activity or levels such that the activity or level of TLR4 is no longer detectable in the presence of the test agent. Such a decrease may be seen in the sample being tested or, for example where the method is carried out in an animal model, in particular tissue from the animal such as in the circulation or other organs such as the liver, kidney or heart.

A test agent that is an antagonist of TLR4 may be a specific or selective antagonist of TLR as described above. For example, the agent may have an effect on other Toll like receptors, such as antagonism of the activity, signaling or expression of one or more other Toll like receptors, that is less than 25%, less than 20%, less than 15%, less than 10%, less than 5%, less than 1%, or less than 0.1% the effect of that agent on the activity, signaling or expression of TLR4.

Levels or amounts of TLR4 may be measured by assessing expression of the TLR4 gene. Gene expression may be assessed by looking at mRNA production or levels or at protein production or levels. Expression products such as mRNA and proteins may be identified or quantified by methods known in the art. Such methods may utilize hybridization to specifically identify the mRNA of interest. For example such methods may involve PCR or real-time PCR approaches. Methods to identify or quantify a protein of interest may involve the use of antibodies that bind that protein. For example, such methods may involve western blotting. Regulation of TLR4 gene expression may be compared in the presence and absence of a test agent. Thus test agents can be identified that decrease TLR4 gene expression compared to the level seen in the absence of the test agent. Such test agents may be suitable antagonists of TLR4 in accordance with the invention.

The screening methods may assess the activity of TLR4. For example, such a method may be carried out using peripheral blood mononuclear cells. Such cells will produce cytokines such as TNF-α, IL-6, IFN-β, IL-1β and IL-8 on response to stimulation with, for example, lipopolysaccharide (LPS). A screening method may therefore comprise combining peripheral blood mononuclear cells with the test agent or a vehicle and adding LPS. The cells may then be incubated for an amount of time (e.g., 24 hours) to allow the production of pro-inflammatory mediators such as cytokines. The level of cytokines such as TNF-α, IL-6, IFN-β, IL-1β and IL-8 produced by the cells in that time period can then be assessed. If the test agent has anti-TLR4 properties, then the production of such cytokines should be reduced compared to the vehicle-treated cells.

Further tests may also be carried out in order to confirm that the test agent is suitable for use in the claimed methods. For example, as explained above, a suitable antagonist of TLR4 should be capable of reducing the deleterious consequences of pro-inflammatory mediator production (also commonly referred to as a cytokine storm) and vascular leakage. The screening methods of the invention may therefore incorporate further steps, such as those discussed above, which involve assessing the effect of the test agent in an animal with such production of pro-inflammatory mediator and vascular leakage (e.g., one infected with a Flavivirus) and comparing that effect with that seen in the absence the test agent. A suitable TLR4 antagonist will be capable of ameliorating at least some of the effects of the Flavivirus infection in the test animal.

2.4 Ancillary Anti-Flavivirus Agents

As indicated, compounds according to the present invention may be administered alone or in combination with other agents (also referred to herein as “ancillary agents”), especially including other compounds of the present invention or compounds which are not TLR4 antagonists and are otherwise disclosed as being useful for the treatment of Flavivirus infections including Dengue virus, Japanese encephalitis virus, Yellow fever virus, Murray Valley encephalitis virus, West Nile virus, Tick-borne encephalitis virus, St Louis encephalitis virus, Alfuy virus, Koutango virus, Kunjin virus, Cacipacore virus, and Yaounde virus and related Flavivirus infections, such as those relevant compounds and compositions which are disclosed in the following United States patents, which are incorporated by reference herein: U.S. Pat. Nos. 5,922,757; 5,830,894; 5,821,242; 5,610,054; 5,532,215; 5,491,135; 5,179,084; 4,902,720; 4,898,888; 4,880,784; 5,929,038; 5,922,857; 5,914,400; 5,922,711; 5,922,694; 5,916,589; 5,912,356; 5,912,265; 5,905,070; 5,892,060; 5,892,052; 5,892,025; 5,883,116; 5,883,113; 5,883,098; 5,880,141; 5,880,106; 5,876,984; 5,874,413; 5,869,522; 5,863,921; 5,863,918; 5,863,905; 5,861,403; 5,852,027; 5,849,800; 5,849,696; 5,847,172; 5,627,160; 5,561,120; 5,631,239; 5,830,898; 5,827,727; 5,830,881 and 5,837,871, among others as well as U.S. Pat. App. Pub. Nos. 2009/0130123, 2014/0170186 and 2014/0248336. In specific embodiments, ancillary anti-Flavivirus agents that are useful in combination with TLR4 antagonists include antivirals and vaccines as well as agents that alleviate the symptoms of Flavivirus infection or prevent secondary infections such as antibiotics used to prevent pneumonia and urinary tract infections, anticonvulsants for seizure control, anti-nausea medicaments, mannitol, interferon alpha (e.g., interferon alpha 2a and interferon alpha 2b), interferon beta (e.g., interferon beta 1a and interferon beta 1b), or nucleic acid constructs from which interferons are expressible, antibody therapy or any combination thereof.

Ancillary anti-Flavivirus agents may be used in combination with TLR4 antagonists for their additive activity or treatment profile Flavivirus infections and, in certain instances, for their synergistic effects in combination with compounds of the present invention.

When combination therapy is desired, the TLR4 antagonist is administered separately, simultaneously or sequentially with ancillary agent. In some embodiments, this may be achieved by administering a single composition or pharmacological formulation that includes both types of agent, or by administering two separate compositions or formulations at the same time, wherein one composition includes the TLR4 antagonist and the ancillary agent. In other embodiments, the treatment with the TLR4 antagonist may precede or follow the treatment with the ancillary agent by intervals ranging from minutes to days. In embodiments where the TLR4 antagonist is applied separately to the ancillary agent, one would generally ensure that a significant period of time did not expire between the time of each delivery, such that the TLR4 antagonist would still be able to exert an advantageously combined effect on inhibiting a TLR4-mediated effect including inhibiting production of pro-inflammatory mediators by cells (e.g., an immune cell such as but not limited to a macrophage or monocyte, or an endothelial cell) with the ancillary agent, and in particular, to maintain or enhance a subject's capacity to reverse or inhibit the development of disease or symptoms associated with Flavivirus infection. In some situations, one may administer both modalities within about 1-12 hours of each other and, more suitably, within about 2-6 hours of each other. In some situations, it may be desirable to extend the time period for treatment significantly, however, where several hours (2, 3, 4, 5, 6 or 7) to several days (1, 2, 3, 4, 5, 6, 7 or 8) lapse between the respective administrations. In specific embodiments, the antiviral agent is administered prior to the administration of the TLR4 antagonist, suitably at an early stage of infection and/or before the onset of severe disease. In these Instances, the TLR4 antagonist is suitably administered also before the onset of severe disease.

It is conceivable that more than one administration of either the TLR4 antagonist or the ancillary agent will be desired. Various combinations may be employed, where the TLR4 antagonist is “A” and the ancillary agent is “B”, as exemplified below:

A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A/AA/B B/A/A/A A/B/A/A A/A/B/A A/B/B/B B/A/B/B B/B/A/B.

2.5 Compositions

In accordance with the present invention, it is proposed that TLR4 antagonists, whether alone or in combination with ancillary anti-Flavivirus agents, can be used to inhibit production of pro-inflammatory mediators, including pro-inflammatory cytokines, and reducing the sequelae of that production including inhibiting or ameliorating vascular leakage, and more particularly, to treat or prevent Flavivirus infections and their symptoms, including in severe disease.

TLR4 antagonists and optionally the ancillary anti-Flavivirus agents can be administered either by themselves or with a pharmaceutically acceptable carrier. Thus, in some embodiments, the compositions of the invention are administered in pharmaceutically acceptable solutions, which may routinely contain pharmaceutically acceptable concentrations of salt, buffering agents, preservatives, compatible carriers, adjuvants, and optionally other therapeutic ingredients. Depending on the specific conditions being treated, the compositions may be administered systemically or locally. Techniques for formulation and administration may be found in “Remington's Pharmaceutical Sciences,” Mack Publishing Co., Easton, Pa., latest edition. The compositions may be administered orally, topically, transdermally, parenterally, subcutaneously, intravenously (e.g., hepatic vein), Intramuscularly, intraperitoneally, intracavitary, by intravesical instillation, intranasally, intraocularty, intraarterially, intralesionally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

TLR4 antagonists and optionally the ancillary anti-Flavivirus agents may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

TLR4 antagonists and optionally the ancillary anti-Flavivirus agents may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. hi general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for Injectable use Include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

TLR4 antagonists and optionally the ancillary anti-Flavivirus agents may also be administered directly to the airways in the form of an aerosol. For use as aerosols, the inhibitors of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Persons of skill in the art are readily able to test and assess optimal dosage schedules based on the balance of efficacy and any undesirable side effects. The optimal dosage of each type of inhibitor will vary, of course, and the minimal effective dose will be administered for therapeutic regimen.

In order that the invention may be readily understood and put into practical effect, particular preferred embodiments will now be described by way of the following non-limiting example.

### Example

### Dengue Virus NS1 is a Viral Toxin that Activates Cells Via TLR4 and Disrupts Endothelial Cell Monolayer Integrity

### NS1 Activates Human and Mouse Immune Cells

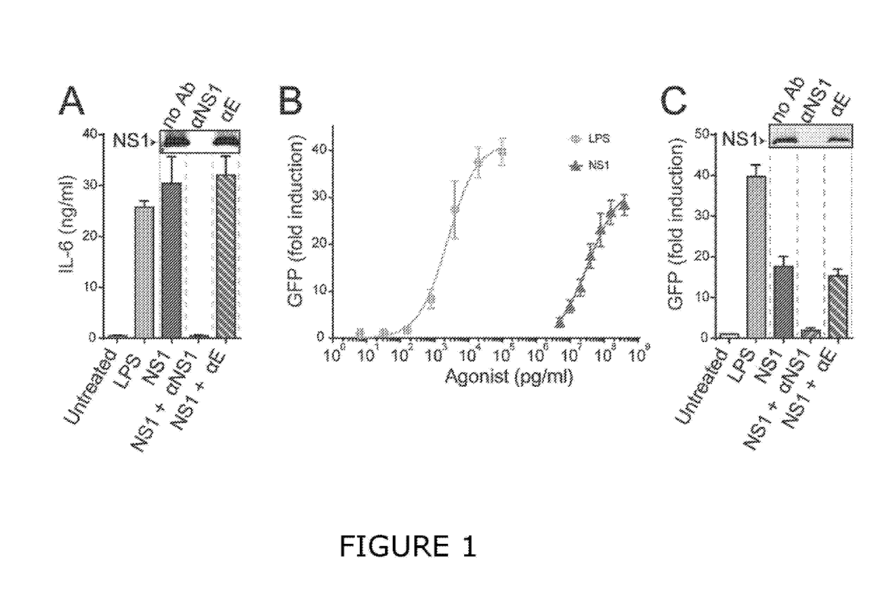

Circulating high levels of sNS1 are known to correlate with progression to severe disease (13, 16). Whether these levels are simply a surrogate marker of infection or play a direct role in disease severity is not known. The present inventors hypothesized that circulating NS1 directly activates innate immune cells, contributing to the induction of inflammatory cytokines in severe dengue. Recombinant, hexameric sNS1 was purified from media harvests of S2 cells stably expressing DENV-2 NS1. The purity of NS1 after immunoaffinity chromatography was assessed by SDS-PAGE and size exclusion chromatography. Throughout this study LPS was used as a positive control. Purified sNS1 and LPS both strongly induced the secretion of IL-6 from peripheral blood mononuclear cells (PBMCs) (FIG. 1A), suggesting NS1 behaves as a PAMP. To provide evidence that NS1 itself was responsible, the activity of the sNS1 preparation was investigated before and after immuno-depletion using anti-NS1 antibody or an isotype-matched control antibody specific for the dengue virus envelope protein E (FIG. 1A). NS1-depletion prevented IL-6 induction, whereas the control antibody neither depleted NS1 nor abrogated activity.

A mouse model was then used to facilitate characterization of the response mechanism. Recognition of many PAMPs by innate immune cells triggers the activation of the transcription factor nuclear factor-kappa B (NF-κB), which induces an array of pro-inflammatory cytokines and chemokines. The present inventors investigated whether NS1 treatment activated NF-κB using the RAW264.7 mouse macrophage cell line stably transfected with the NF-κB-responsive ELAM promoter driving GFP (17). Cells exposed to purified NS1 or LPS displayed increased GFP reporter-gene activity (FIG. 18B) and the effect of NS1 was abolished by prior immuno-depletion (FIG. 1C).

### LPS-Inhibitory Polmyxin B does not Affect NS1 Stimulatory Activity

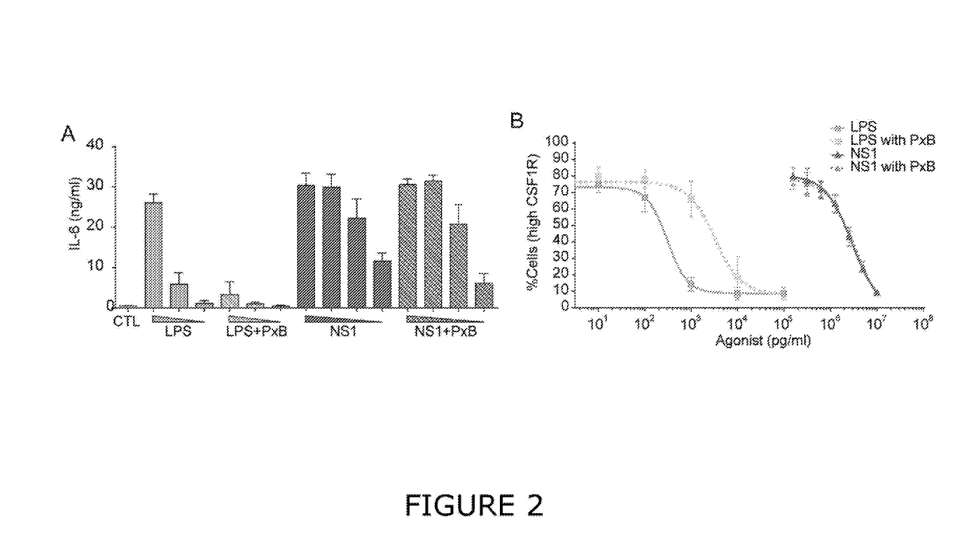

In defining novel PAMPs, the elimination of bacterial contaminants from candidate PAMP preparations is critical. The immune-depletion results above demonstrate that either NS1 or a factor bound to NS1 activates NF-κB and cytokine release in mouse and human cells respectively. Since hexameric NS1 is a lipid-binding protein (18), it is conceivable that the insect (S2) cell-derived NS1 used in this study could have bound trace amounts of bacterial lipids such as LPS during the purification process. To test the possibility that the NS1 preparation contains contaminating LPS, the induction of IL-6 in PBMCs was examined using NS1 pretreated with the LPS-binding antibiotic polymyxin B. Polymyxin B inhibited the response to concentrations of LPS ≤10 ng/mL, whilst the NS1-mediated response was unaffected (FIG. 2A). To examine this in mouse cells, the present inventors measured down-modulation of the receptor for the macrophage growth factor, colony stimulating factor-1 (CSF1). Cell surface CSF1 receptor (CSF1R) is increased on CSF1-starved bone marrow-derived macrophages (BMM), and rapidly lost in response to TLR agonists including LPS, making a convenient and sensitive assay for TLR agonists (19). Both LPS and NS1 down-modulated surface CSF1R, however only the LPS response was sensitive to polymyxin B (FIG. 2B). Thus, LPS contamination is not a likely explanation for the NS1 response of mouse or human cells.

### NS1 Activity is not Due to a Co-Purifying Contaminant

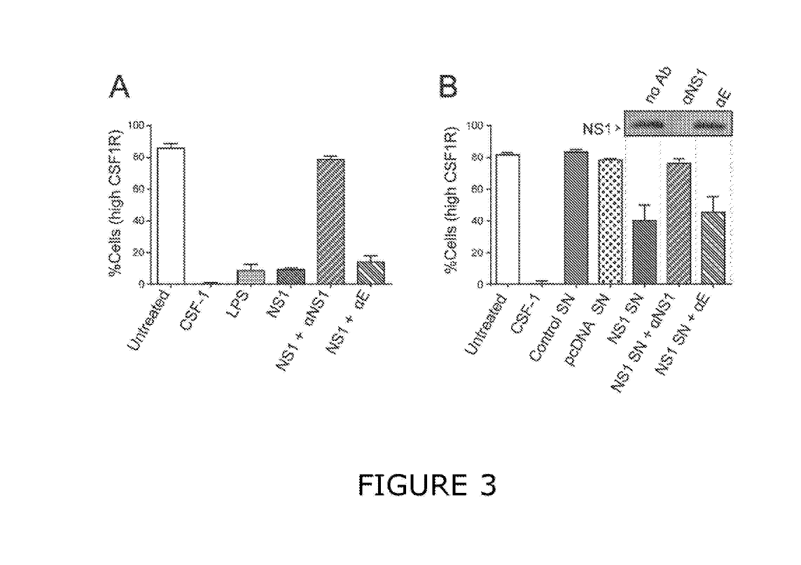

Results of immuno-depletion and polymyxin B experiments above reduce the likelihood that a contaminant is responsible for the activity of NS1 preparations. However, the possibility remained that the active component was a non-LPS NS1-binding contaminant, or LPS which was inaccessible to polymyxin B. The S2-derived NS1 had been affinity purified, and to eliminate concerns about introduction of bacterial contaminants during the purification process, the activity of NS1 expressed by mammalian cells was assessed, without purification. As expected, the S2-derived NS1 caused loss of CSF1R, and this was prevented by NS1 immuno-depletion (FIG. 3A). Medium derived from NS1 expression vector-transfected CHO cells also reproducibly reduced surface CSF1R levels (FIG. 3B). Media from untransfected or empty vector-transfected CHO cells had no effect on CSF1R levels. NS1 immuno-depletion prevented activity of the CHO cell-expressed NS1, whereas a control antibody had no effect. There is no route for microbial product contamination in the chemically defined, serum free tissue culture reagents used in the CHO cell expression, and the control medium samples had no activity. Taken together, it was concluded that the NS1 lipoprotein, rather than a microbial contaminant in the NS1 preparations has immunostimulatory activity.

### NS1 Protein Induces Pro-Inflammatory Cytokines in Mouse Bone Marrow Derived Macrophages and Human PBMCs

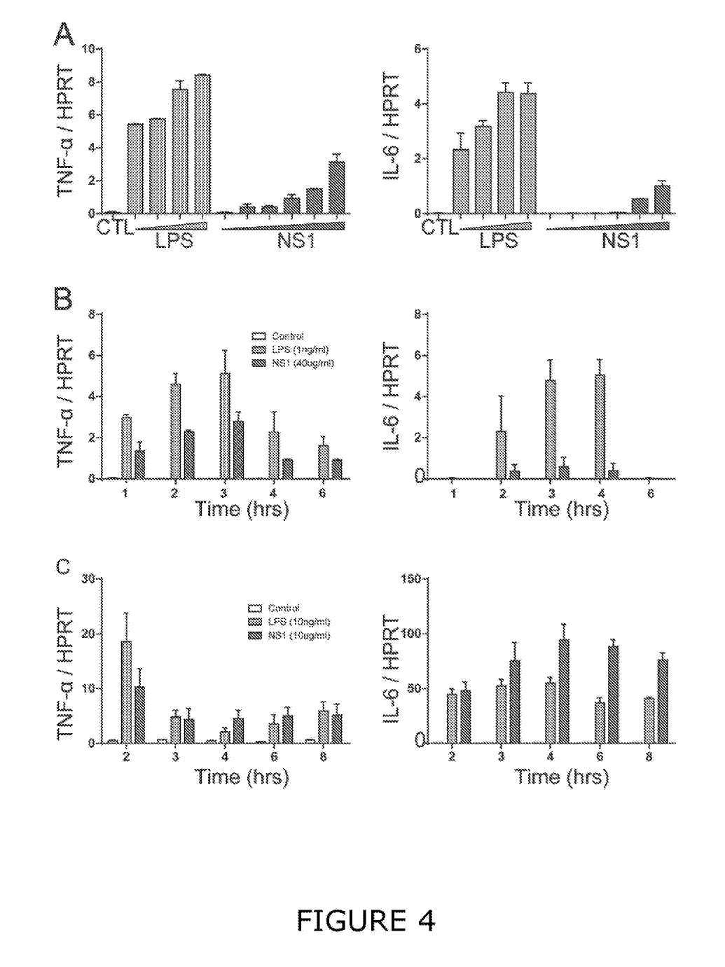

Pro-inflammatory cytokines such as TNF-α, IFN-γ, IL-6, IL-1β and MIF, and chemokines such as IL-8, IP-10 and MCP-1 are induced during the acute phase of dengue infection with levels correlating with disease severity (2, 20, 21). To investigate whether cellular activation by circulating NS1 could contribute to cytokine and chemokine production, mRNA expression was measured in mouse BMM and human PBMC after treatment with NS1. NS1 induced a dose-dependent increase in the levels of mRNA for TNF-α, IL-6, IFN-β, IL-1β, and IL-12 (FIG. 4A), in mouse BMM although this had not reached a maximal response at 40 μg/mL NS1. NS1 induced these genes in a similar time course to LPS (FIG. 4B). Although the response of mouse BMMs to physiologically-relevant concentrations of NS1 was much lower than the response to LPS, both agents produced strong induction of cytokine mRNAs in human PBMCs, with NS1 being a particularly potent inducer of IL-6 and IL-1 β genes (FIG. 4C). TNF-α, IL-6, IFN-β, IL-1β and IL-8 genes were still well expressed after 8 hours, IL-12 was induced at a later time than other genes, and IP-10 and MCP-1 showed modest induction by both agents (FIG. 4C).

### NS1 is Recognized Via TLR4

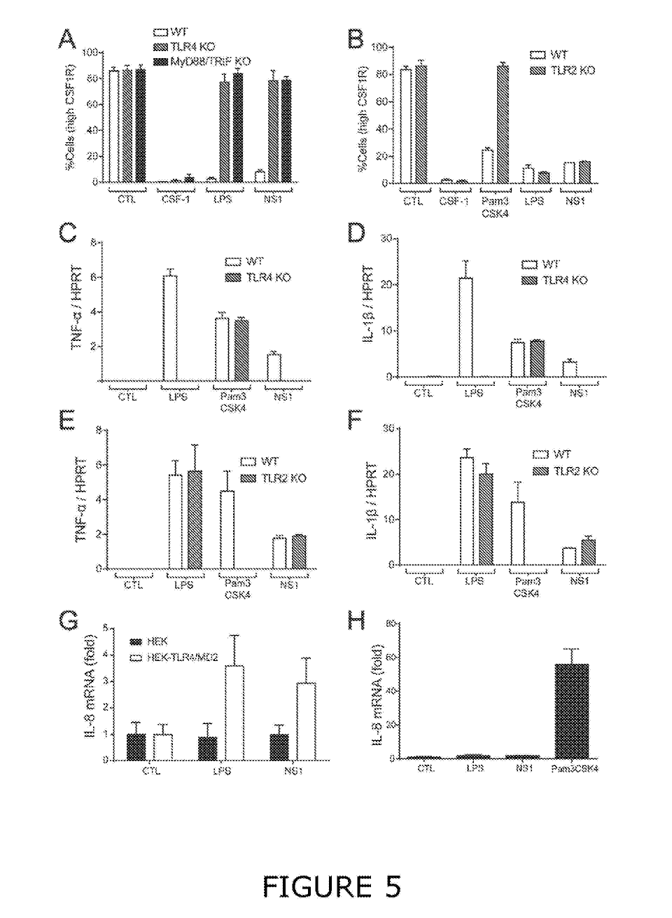

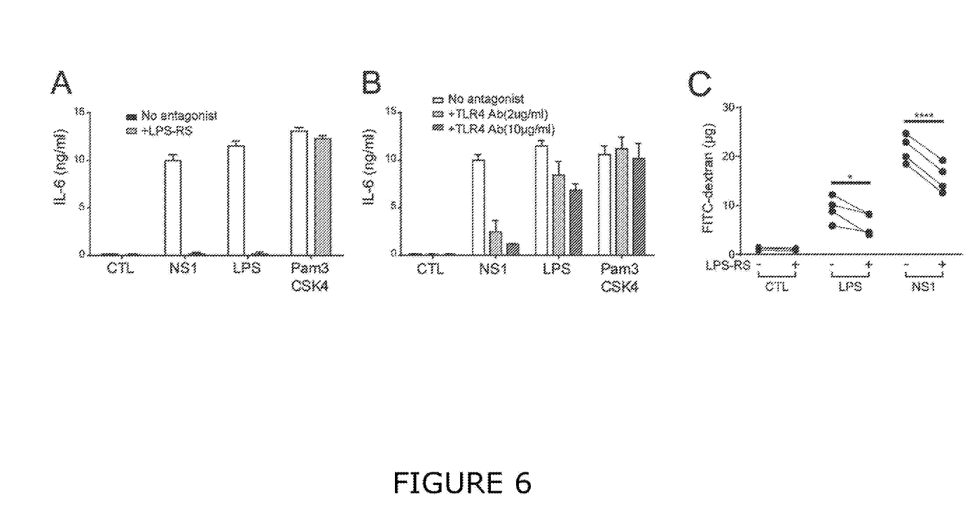

The present inventors reasoned that TLRs are the most likely candidates for cell surface receptors that recognize extracellular NS1 and mediate activation of NF-κB and cytokine induction. Although 12 and 10 TLR family members have been identified in mice and humans, respectively, only TLR2 and TLR4 have been observed to interact with viral proteins. To determine whether NS1 acts via a TLR, the ability of NS1 to down-modulate CSF1R was examined on BMMs from mice lacking either individual TLRs, or TLR signaling adapters MyD88 and TRIF. Both tlr4^{-/-} and MyD88^{-/-}/trif^{-/-} macrophages lacked responses to LPS and NS1, but responses were intact in tlr2^{-/-} cells. Pam₃CSK₄ was used as a control stimulus for TLR2 (FIGS. 5A and B). tlr4^{-/-} BMM also failed to respond to NS1 with increased TNF-α, IL-1β or other cytokine mRNAs (FIGS. 5C and D), but these responses were intact in tlr2^{-/-} cells (FIGS. 5E and F). To confirm that human TLR4 recognizes NS1, IL-8 mRNA was measured in HEK293 cells ectopically expressing TLR4/MD2 or TLR2, stimulated with NS1, LPS or Pam₃CSK₄. A response to NS1 and LPS was seen in cells expressing TLR4/MD2 (FIG. 5G) but not TLR2 (FIG. 5H), consistent with the results from gene-targeted knock-out mouse BMMs. To demonstrate the involvement of TLR4 in human PBMC responses to NS1, the present inventors took advantage of a naturally occurring TLR4 antagonist. The pattern of lipid A acylation of Rhodobacter sphaeroides LPS (LPS-RS) renders it non-stimulatory and inhibitory to active LPS. It has been suggested to antagonize LPS activity by competitive binding to both TLR4/MD-2 complex and serum LPS-binding protein (LBP) (22, 23). Pre-treatment of PBMCs with LPS-RS completely inhibited the activities of LPS and NS1 but not Pam₃CSK₄ (FIG. 6A), confirming a requirement for human TLR4 in cytokine induction by NS1. Interestingly, a TLR4 blocking antibody was more effective at reducing the response to NS1 than it was to LPS, perhaps reflecting a greater ability to sterically block the larger NS1 macromolecule (FIG. 68).

### NS1 has Direct Effects on Endothelial Cells

Vascular leakage leading to shock is one of the hallmarks of severe dengue infection. NS1 has been suggested to contribute to vascular leak (24), and might have direct effects on endothelial cells, which can express TLR4 (25). The present inventors analyzed the integrity of HMEC-1 monolayers grown in transwell cultures following exposure to NS1 or LPS, and found that permeability to FITC-dextran (4 kDa) increased over time when compared to the untreated cell monolayer (FIG. 6C). Treatment with LPS-RS resulted in a reproducible but incomplete inhibition of the response to both LPS and NS1. Together with earlier work showing that TLR4 mediates endothelial monolayer leak in response to LPS (25) this implies that in addition to mediating cytokine induction from monocytes/macrophages, NS1 recognition by TLR4 on endothelial cells directly contributes to vascular leak.

### NS1 and TLR4 Co-Localize on the Cell Surface

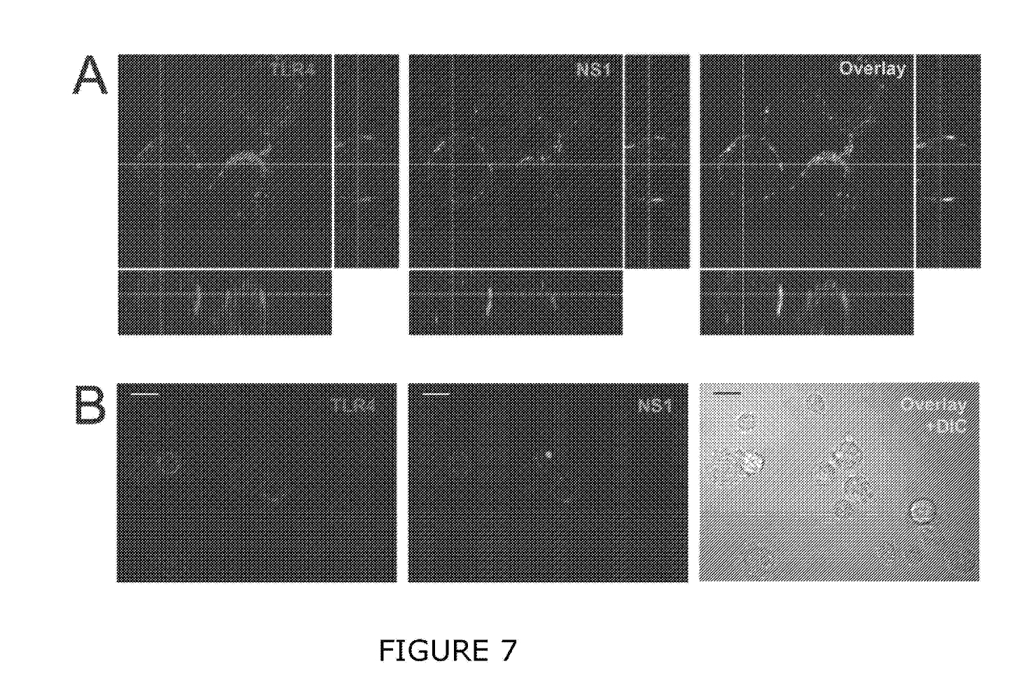

In order to visualize the interaction between cell surface TLR4 and NS1 co-localization experiments were performed using human PBMCs. PBMCs were allowed to adhere to coverslips for 2 hours, and then exposed to exogenous NS1 at physiological levels (10 μg/mL) to allow receptor interaction. After subsequent washing and fixation, cell surface TLR4 and NS1 were co-stained using antibody probes prior to optical sectioning by confocal microscopy. Although there was not complete co-localization, a prominent overlap in the signal for TLR4 and NS1 was observed on the surface of cells (FIG. 7A). This is particularly clear in the z stack analysis, which showed clear association in isolated regions of the membrane (FIG. 7A). Amongst PBMCs, monocytes are the most abundant cells with readily detectable TLR4 expression, with lymphocytes expressing very little TLR4 mRNA (26). The adherent PBMC were a mixture of cell types, and interestingly, the cells without observable surface TLR4 were devoid of significant NS1 binding (FIG. 7B).

Discussion

In this study the present inventors have shown that the hexameric, secreted form of dengue virus NS1 directly activates mouse macrophages and human PBMCs via TLR4 with the consequent release of pro-inflammatory cytokines. This finding suggests that NS1 plays a contributing role in triggering the cytokine storm that has been proposed as being responsible for the vascular leak and shock in severe dengue disease (2). In addition, NS1 was found to directly impair endothelial cell monolayer integrity in an in vitro model of leak. These findings provide a mechanistic framework for the in vivo activity of sNS1, and together provide a new paradigm for dengue disease pathogenesis, with circulating NS1 playing a major role.

TLR4 is expressed on several cell types including monocytes, macrophages and endothelial cells. Recognition of TLR4 by LPS is well studied, with serum LPS binding protein (LBP) transferring LPS to CD14, either in its GPI-anchored form on myeloid cells or as a soluble species found in serum. CD14 in turn delivers LPS monomers to a complex of TLR4 and MD-2. The crystal structure of this complex showed that the predominant interaction with LPS is mediated by MD-2 (27) with the acyl chains of the central lipid A core of LPS being buried in a hydrophobic pocket in MD-2. Dimerization of TLR4-MD2-LPS complexes promotes recruitment of adapter proteins to the cytosolic domains and subsequent downstream signaling. Antagonists of human TLR4 signaling, such as LPS-RS, and its synthetic counterpart eritoran bind within the MD-2 hydrophobic pocket, but lack a critical acyl chain required for TLR4-MD-2 complex dimerization (28). The present inventors found that LPS-RS was also a highly potent antagonist of NS1-mediated induction of cytokines, suggesting that NS1 also interacts with MD-2.

In addition to LPS, TLR4 is reported to mediate responses to many other structurally and chemically diverse ligands of microbial and host origin. Putative viral ligands of TLR4 include RSV F protein (29), mouse mammary tumor virus envelope protein (30), and Ebola virus glycoprotein (31). Although a number of endogenous “alarmin” molecules, such as hsp70 and HMGB1 are also reported to stimulate TLR4, it has been proposed that many of these actually either bind and present LPS to cells, or sensitize cells to respond to LPS (32). The same issues potentially apply to microbial molecules identified as TLR4 agonists and so the present inventors used several approaches to confirm that NS1 lipoprotein alone mediates the observed cellular activation. The activity was removed by anti-NS1 specific immuno-depletion, and was resistant to polymyxin B treatment. Importantly, TLR4 stimulatory activity was retained in media harvests of mammalian cells expressing NS1, avoiding the possibility of introduction of endotoxin during column purification. As noted above, NS1 is secreted as a lipid-carrying complex (18, 33), and the relative contribution of NS1 protein and associated lipid moieties to TLR4 stimulation remains to be established. However, the TLR4 stimulatory activity of RSV F protein, like NS1, is inhibited by LPS-RS, with the hydrophobic N terminal region of the F protein shown to be responsible for interaction with the TLR4-MD-2 complex (29). The recent crystal structure determination of NS1 dimer and hexamer assemblies has revealed exposed hydrophobic domains (10) and the possibility that these may be responsible for the NS1 activity we have observed is currently under investigation.

NS1 was shown to induce an array of pro-inflammatory cytokines and chemokines. In dengue patients, levels of TNF-α, IL-1β, IL-6, IFN-γ, IL-8 and MCP-1 all correlate with disease severity (2, 20, 21). Whilst TLR4 recognition of NS1 may provide early priming of innate immune activation, which promotes viral clearance, excessive cytokine induction by the high levels of NS1 associated with severe disease (13) may also contribute to pathology. Increased vascular permeability is the primary manifestation of severe dengue and so the mechanism of endothelial cell response to dengue infection is of great interest. Many of the cytokines induced by NS1 are implicated in the perturbation of endothelial cell barrier function and so may promote vascular leak in severe dengue infection. For example, IL-11 in combination with TNF-α and IFN-γ affects the barrier function of endothelial cells (34) while IL-8 and MCP-1 are both reported to alter endothelial tight Junctions (35, 36). In the context of dengue infection, TNF-α has been suggested as a mediator of dengue-associated hemorrhage (37), and a recent report revealed that TNF-α produced in the dengue-infected mouse model induced apoptosis in endothelial cells (38). In addition to its role in induction of cytokines, NS1 may also have a direct effect on endothelial cell tight junctions. The loss of in vitro endothelial monolayer integrity observed here after only 2 hours of exposure to NS1 is most likely a direct effect of NS1 on endothelial cell signaling pathways. Indeed, LPS has been shown to directly induce endothelial leak via TLR4, with involvement of calcium Influx (39) and tyrosine kinase pathways (40).

It has previously been shown that circulating levels of NS1 early during the acute stage of secondary infection, from days 2-4 after the onset of symptoms, correlate with progression to severe disease (11). The latter typically occurs from days 4-6 post symptom onset and at a time when virus levels are in rapid decline (13). While NS1 levels have been accepted as a surrogate marker for viral load, the present inventors reported earlier that the clearance of NS1 and virus follow different kinetics with circulating NS1 being cleared more than a day later than virus (11). Consequently, mean plasma levels of circulating NS1 were found to still be higher for DHF/DSS patients at the time of severe vascular leak onset when compared to DF patients (11). It is also likely that circulating levels of NS1 are not a true reflection of the levels of NS1 adsorbed and sequestered on endothelial cell surfaces over time. The finding by Beatty et al., (personal communication) of maximal leak and morbidity 3 days after NS1 inoculation in an in vivo mouse model is consistent with this hypothesis and provides further support for a role for NS1 is disease exacerbation.

In summary, the present inventors show NS1 recognition by TLR4 stimulates innate immune cytokine and chemokine production, and disrupts endothelial monolayer integrity at clinically relevant, physiological levels. They further show that NS1 stimulation of cytokine production can be completely inhibited using the TLR4 antagonist LPS-RS. This finding offers a route to therapeutic intervention with the possible re-purposing of existing sepsis drug candidates. Overall, the parallels between NS1 and LPS are compelling, and the present inventors suggest that NS1 be considered a viral counterpart of this endotoxin. LPS in bacterial sepsis contributes to a cytokine storm, vascular permeability and septic shock, and NS1 may generate an analogous aseptic shock in dengue virus infected patients.

### Materials and Methods

Materials

Ultrapure LPS from E. coli 0111:B4 strain, LPS-RS and polymyxin B were obtained from InvivoGen, Pam3CSK4 kindly provided by Kelly Smith (Dept. of Pathology, University of Washington, Seattle Wash., USA) was originally purchased from Roche. The anti-TLR4 antibody (HTA125) was purchased from Abcam.

Cell Culture

The murine macrophage-like cell line RAW264.7 stably transfected with the human ELAM promoter driving GFP expression has been described (17). BMM were obtained by ex vivo differentiation of mouse bone marrow progenitors in the presence of CSF1 for 7 days (19). Use of mice was approved by the University of Queensland Animal Ethics Committee. PBMCs were obtained from healthy volunteers under approval from the University of Queensland Medical Research Ethics Committee. Assessment by flow cytometry of NF-κB-responsive ELAM promoter activity, and CSF1R down-modulation as responses to TLR ligation have been described (17, 19). HEK293 cells, stably transfected with hTLR2 or hTLR4/MD-2 were obtained from Doug Golenbock, University of Massachusetts. HMEC-1 cells were grown in MCDB 131 medium supplemented with 10% FCS, 1 μg/mL of hydrocortisone and 10 ng/mL of human epidermal growth factor.

Generation and Purification of DENV-2 NS1

Recombinant NS1 was expressed by stably transfected Drosophila S2 cells. The protein was affinity purified from culture medium using 2A5.1 anti-NS1 monoclonal antibody. NS1 protein was also transiently expressed in Chinese Hamster Ovary cells (CHO). Transfection complex was washed away after 4 hours. The medium was harvested at day 2 post-transfection and concentrated using a 100 kDa cut-off spin column (Millipore).

Determination of Protein Concentration

Purified protein concentrations were determined using a BCA assay Kit (Pierce). NS1 protein expression in the CHO expression system was determined by capture-ELISA as described previously (15), with the following modifications: the inclusion of NS1 standards (purified recombinant NS1 (0.3 ng/mL to 1250 ng/mL) and 100 μg/mL TMB and sulfuric acid were used for color detection.

Depletion of NS1 from Stably Transfected Cell Medium

Mouse monoclonal anti-NS1 antibody (1H7) and anti-E antibody (3H5), purified from ascites were bound to protein G Dynabeads (Life Technologies). Purified NS1 from S2 cells and CHO medium were mixed with beads and incubated for 2 hours at room temperature. The supernatants were collected following magnet-mediated bead separation, as NS1-depleted and control-depleted samples. The efficiency of NS1 depletion was confirmed by western blot using rabbit anti-NS1 antibody.

NF-κB-Driven Promoter Reporter Assay

RAW264.7 macrophages expressing the ELAM promoter driving GFP (17) were seeded at 5×10⁵ cells/well on sterilin 25 well bacteriological plates in 0.5 mL prior to stimulation with indicated amount of LPS or purified NS1. After 6 hours cells were harvested by washing with PBS three times and harvested in Dulbecco's PBS containing 0.02% NaN₃. GFP expression was analyzed by flow cytometry on a BD Accuri 6.

CSF-1R Down-Modulation Assay

BMMs were cultured overnight in complete RPMI1640 medium without CSF1. Cells were subsequently treated with NS1, LPS, Pam₃CysK₄ or immune-depleted NS1 for 1 hour in 1.5 mL microcentrifuge tube. The cells were washed using PBS containing 0.02% NaN₃. The cells were centrifuged at 500×g for 5 min and stained with CD115 antibody conjugated with RPE (Serotec) for 1 hour at 4° C. at 1:100 dilution in PBS containing 1% BSA and 1% FCS. The cells were centrifuged, washed using PBS containing 1% BSA and 1% FCS, resuspended in PBS and monitored for CSF-1R expression on the cell surface by flow cytometry.

Analysis of mRNA by Quantitative Real-Time Reverse Transcriptase PCR (qRT-PCR)

Mouse BMMs (with CSF-1), or human PBMCs were seeded at 2×10⁶ and 1×10⁶ cells per well in 6 cell plates 24 hours prior to stimulation with the indicated amount of NS1 or TLR ligands. HEK293 cells and HEK293-TLR cell lines were plated overnight at 1×10⁶ per well in 1 mL medium in a 12-well plate, treated on the following day and harvested at 3 hours post-treatment. Total RNAs were prepared with RNeasy RNA Mini Kit (Qiagen) and the cDNA was reverse transcribed from 2 μg of total RNA with random hexamer primer using a ThermoScript RT Kit (Invitrogen). qPCR was then carried out using specific primers set for mouse TNF-α, IL-1β, IL-6, IL-12, IFN-β and human TNF-α, IFN-γ, IFN-β, IL-6, IL-1β, IL-8, IP-10 and MCP-1 with SYBER©green PCR MasterMix (Life Technologies) according to manufacturer's recommendations. Analysis was done by Applied Biosystems ViiA™ 7 Real-time PCR system. HPRT gene expression was used as the reference for both mouse and human cells.

Detection of IL-6 and IL-8 Production Using ELISA

Human PBMCs from healthy donors were seeded at 1×10⁶ cells per well in 12 well plates prior to incubation with NS1 or LPS. In some experiments, LPS-RS was pre-incubated with cells for 30 min. The media of treated cells were harvested and centrifuged at 1000 g for 5 min. The level of IL-6 production was quantitated ELISA (R & D Systems) according to the protocol recommended by the manufacturer.

Indirect Immunofluorescence and Confocal Microscopy

Human PBMCs were isolated and seeded onto coverslips. After 2 hours of incubation, unbound cells were removed by washing twice with complete medium. NS1 (10 μg/mL) was added and incubated for 45 min at 37° C. Cells were washed three times with PBS, fixed with 4% paraformaldehyde in PBS for 10 min and incubated with PBS containing 0.05% non-fat dry milk and 0.05% of Tween-20. The cells were then stained with rabbit anti-NS1 polyclonal antibody and mouse anti-TLR4 monoclonal antibody (HTA125) in 0.05% of PBS-T for 1 h at 37° C. Coverslips were washed three times in 0.05% of PBS-T and incubated with goat anti-rabbit Alexa Fluor 488-conjugated or goat anti-mouse Alexa Fluor 555-conjugated secondary antibodies (Ufe Technologies) for 1 h at 37° C. and washed three times in 0.05% of PBS-T. The cells were examined using ZEISS 510 and 710 META microscopy at 100× magnification. The images were analyzed using Image) version 10.

HMEC-1 Monolayer Permeability Measurement

HMEC-1 were grown to confluency on collagen-coated transwell plates with 0.4 μm pore size membranes (Corning). Cells were seeded at 1×10⁵ cells per well, and after 48 hours the medium was removed and fresh medium containing LPS or NS1 were added and cells were incubated for 2 hours. Monolayer permeability was determined by measuring the flux of fluorescein isothiocyanate dextran (FITC-dextran, 4 kDa) (Sigma-Aldrich) from the apical to basolateral chambers. Medium was removed and inserts were transferred into new transwells with fresh medium containing FITC-dextran (100 μg/mL) for 1 hour. The FITC-dextran concentration was determined using excitation at 485 nm and fluorescence emission at 520 nm, and generating a standard curve.

The disclosure of every patent, patent application, and publication cited herein is hereby Incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. S. Bhatt, P. W. Gething, O. J. Brady, J. P. Messina, A. W. Farlow, C. L. Moyes, J. M. Drake, J. S. Brownstein, A. G. Hoen, O. Sankoh, M. F. Myers, D. B. George, T. Jaenisch, G. R. Wint, C. P. Simmons, T. W. Scott, J. J. Farrar, S. I. Hay, The global distribution and burden of dengue. Nature 496, 504-507 (2013).
2. U. C. Chaturvedi, R. Agarwal, E. A. Ebishbishi, A. S. Mustafa, Cytokine cascade in dengue hemorrhagic fever: implications for pathogenesis. FEMS Immunol Med Microbiol 28, 183-188 (2000).
3. S. B. Halstead, Dengue. Lancet 370, 1644-1652 (2007).
4. K. Boonnak, B. M. Slike, T. H. Burgess, R. M. Mason, S. J. Wu, P. Sun, K. Porter, I. F. Rudiman, D. Yuwono, P. Puthavathana, M. A. Marovich, Role of dendritic cells in antibody-dependent enhancement of dengue virus infection. J Virol 82, 3939-3951 (2008).
5. M. G. Guzman, M. Alvarez, S. B. Halstead, Secondary infection as a risk factor for dengue hemorrhagic fever/dengue shock syndrome: an historical perspective and role of antibody-dependent enhancement of infection. Arch Virol 158, 1445-1459 (2013).
6. J. Mongkolsapaya, W. Dejnirattisal, X. N. Xu, S. Vasanawathana, N. Tangthawomchaikul, A. Chairunsr, S. Sawasdivorn, T. Duangchinda, T. Dong, S. Rowland-Jones, P. T. Yenchitsomanus, A. McMichael, P. Malasit, G. Screaton, Original antigenic sin and apoptosis in the pathogenesis of dengue hemorrhagic fever. Nat Med 9, 921-927 (2003).
7. A. L. Rothman, Immunity to dengue virus: a tale of original antigenic sin and tropical cytokine storms. Nat Rev Immunol 11, 532-543 (2011).
8. B. E. Martina, P. Koraka, A. D. Osterhaus, Dengue virus pathogenesis: an integrated view. Clin Microbiol Rev 22, 564-581 (2009).
9. D. A. Muller, P. R. Young, The flavivirus NS1 protein: molecular and structural biology, immunology, role in pathogenesis and application as a diagnostic biomarker. Antiviral Res 98, 192-208 (2013).
10. D. L. Akey, W. C. Brown, S. Dutta, J. Konwerski, J. Jose, T. J. Jurkiw, J. DelProposto, C. M. Ogata, G. Skiniotis, R. J. Kuhn, J. L. Smith, Flavivirus NS1 structures reveal surfaces for associations with membranes and the immune system. Science 343, 881-885 (2014).
11. J. M. Mackenzie, M. K. Jones, P. R. Young, Immunolocalization of the dengue virus nonstructural glycoprotein NS1 suggests a role in viral RNA replication. Virology 220, 232-240 (1996).
12. M. G. Jacobs, P. J. Robinson, C. Bletchly, J. M. Mackenzie, P. R. Young, Dengue virus nonstructural protein 1 is expressed in a glycosyl-phosphatidylinositol-linked form that is capable of signal transduction. FASEB J 14, 1603-1610 (2000).
13. D. H. Libraty, P. R. Young, D. Pickering, T. P. Endy, S. Kalayanarooj, S. Green, D. W. Vaughn, A. Nisalak, F. A. Ennis, A. L. Rothman, High circulating levels of the dengue virus nonstructural protein NS1 early in dengue illness correlate with the development of dengue hemorrhagic fever. J Infect Dis 186, 1165-1168 (2002).
14. S. R. Fry, M. Meyer, M. G. Semple, C. P. Simmons, S. D. Sekaran, J. X. Huang, C. McElnea, C. Y. Huang, A. Valks, P. R. Young, M. A. Cooper, The diagnostic sensitivity of dengue rapid test assays is significantly enhanced by using a combined antigen and antibody testing approach. PLoS Negl Trop Dis 5, e1199 (2011).
15. P. R. Young, P. A. Hilditch, C. Bletchly, W. Halloran, An antigen capture enzyme-linked immunosorbent assay reveals high levels of the dengue virus protein NS1 in the sera of infected patients. J Clin Microbiol 38, 1053-1057 (2000).
16. P. Avirutnan, N. Punyadee, S. Noisakran, C. Komoltri, S. Thiemmeca, K. Auethavomanan, A. Jairungsri, R. Kanlaya, N. Tangthawomchaikul, C. Puttikhunt, S. N. Pattanakitsakul, P. T. Yenchitsomanus, J. Mongkolsapaya, W. Kasinrerk, N. Sittisombut, M. Husmann, M. Blettner, S. Vasanawathana, S. Bhakdi, P. Malasit, Vascular leakage in severe dengue virus infections: a potential role for the nonstructural viral protein NS1 and complement. J Infect Dis 193, 1078-1088 (2006).
17. K. J. Stacey, G. R. Young, F. Clark, D. P. Sester, T. L. Roberts, S. Naik, M. J. Sweet, D. A. Hume, The molecular basis for the lack of immunostimulatory activity of vertebrate DNA. J Immunol 170, 3614-3620 (2003).
18. I. Gutsche, F. Coulibaly, J. E. Voss, J. Salmon, J. d'Alayer, M. Ermonval, E. Larquet, P. Charneau, T. Krey, F. Megret, E. Guittet, F. A. Rey, M. Flamand, Secreted dengue virus nonstructural protein NS1 is an atypical barrel-shaped high-density lipoprotein. Proc Natl Acad Sci USA 108, 8003-8008 (2011).
19. D. P. Sester, S. J. Beasley, M. J. Sweet, L. F. Fowles, S. L. Cronau, K. J. Stacey, D. A. Hume, Bacterial/CpG DNA down-modulates colony stimulating factor-1 receptor surface expression on murine bone marrow-derived macrophages with concomitant growth arrest and factor-independent survival. J Immunol 163, 6541-6550 (1999).
20. Y. R. Lee, M. T. Liu, H. Y. Lei, C. C. Liu, J. M. Wu, Y. C. Tung, Y. S. Un, T. M. Yeh, S. H. Chen, H. S. Liu, MCP-1, a highly expressed chemokine in dengue haemorrhagic fever/dengue shock syndrome patients, may cause permeability change, possibly through reduced tight junctions of vascular endothelium cells. J Gen Virol 87, 3623-3630 (2006).
21. A. Rathakrishnan, S. M. Wang, Y. Hu, A. M. Khan, S. Ponnampalavanar, L. C. Lum, R. Manikam, S. D. Sekaran, Cytokine expression profile of dengue patients at different phases of illness. PLoS One 7, e52215 (2012).
22. Y. Aida, K. Kusumoto, K. Nakatomi, H. Takada, M. J. Pabst, K. Maeda, An analogue of lipid A and LPS from Rhodobacter sphaeroides inhibits neutrophil responses to LPS by blocking receptor recognition of LPS and by depleting LPS-binding protein in plasma. J Leukoc Blol 58, 675-682 (1995).
23. A. Teghanemt, D. Zhang, E. N. Levis, J. P. Weiss, T. L. Gioannini, Molecular basis of reduced potency of underacylated endotoxins. J Immunol 175, 4669-4676 (2005).
24. Y. C. Chuang, S. Y. Wang, Y. S. Un, H. R. Chen, T. M. Yeh, Re-evaluation of the pathogenic roles of nonstructural protein 1 and its antibodies during dengue virus infection. J Biomed Sci 20, 42 (2013).
25. P. Gong, D. J. Angelini, S. Yang, G. Xia, A. S. Cross, D. Mann, D. D. Bannerman, S. N. Vogel, S. E. Goldblum, TLR4 signaling is coupled to SRC family kinase activation, tyrosine phosphorylation of zonula adherens proteins, and opening of the paracellular pathway in human lung microvascular endothelia. J Biol Chem 283, 13437-13449 (2008).
36. V. Hornung, S. Rothenfusser, S. Britsch, A. Krug, B. Jahrsdorfer, T. Giese, S. Endres, G. Hartmann, Quantitative expression of toll-like receptor 1-10 mRNA in cellular subsets of human peripheral blood mononuclear cells and sensitivity to CpG oligodeoxynucleotides. J Immunol 168, 4531-4537 (2002).
27. B. S. Park, D. H. Song, H. M. Kim, B. S. Choi, H. Lee, J. O. Lee, The structural basis of lipopolysaccharide recognition by the TLR4-MD-2 complex. Nature 458, 1191-1195 (2009).
28. H. M. Kim, B. S. Park, J. I. Kim, S. E. Kim, J. Lee, S. C. Oh, P. Enkhbayar, N. Matsushima, H. Lee, O. J. Yoo, J. O. Lee, Crystal structure of the TLR4-MD-2 complex with bound endotoxin antagonist Eritoran. Cell 130, 906-917 (2007).
29. P. Rallabhandi, R. L. Phillips, M. S. Boukhvalova, L. M. Pletneva, K. A. Shirey, T. L. Gioannini, 3. P. Weiss, 3. C. Chow, L. D. Hawkins, S. N. Vogel, 3. C. Blanco, Respiratory syncytial virus fusion protein-induced toll-like receptor 4 (TLR4) signaling is inhibited by the TLR4 antagonists Rhodobacter sphaeroides lipopolysaccharide and eritoran (E5564) and requires direct interaction with MD-2. MBio 3, (2012).
30. D. Burzyn, J. C. Rassa, D. Kim, I. Nepomnaschy, S. R. Ross, I. Plazzon, Toll-like receptor 4-dependent activation of dendritic cells by a retrovirus. J Virol 78, 576-584 (2004).
31. A. Okumura, P. M. Pitha, A. Yoshimura, R. N. Harty, Interaction between Ebola virus glycoprotein and host toll-like receptor 4 leads to induction of proinflammatory cytokines and SOCS1. J Virol 84, 27-33 (2010).
32. C. Erridge, Endogenous ligands of TLR2 and TLR4: agonists or assistants? J Leukoc Blol 87, 989-999 (2010).
33. D. A. Muller, M. 3. Landsberg, C. Bletchly, R. Rothnagel, L. Waddington, B. Hankamer, P. R. Young, Structure of the dengue virus glycoprotein non-structural protein 1 by electron microscopy and single-particle analysis. J Gen Virol 93, 771-779 (2012).
34. A. L. Seynhaeve, J. A. Rens, D. Schipper, A. M. Eggermont, T. L Ten Hagen, Exposing endothelial cells to tumor necrosis factor-alpha and peripheral blood mononuclear cells damage endothelial integrity via interleukin-1ss by degradation of vascular endothelial-cadherin. Surgery 155, 545-553 (2014).
35. D. Talavera, A. M. Castillo, M. C. Dominguez, A. E. Gutierrez, I. Meza, IL8 release, tight junction and cytoskeleton dynamic reorganization conducive to permeability increase are induced by dengue virus infection of microvascular endothelial monolayers. J Gen Virol 85, 1801-1813 (2004).
36. M. Baggiolini, P. Loetscher, B. Moser, Interleukin-8 and the chemokine family. Int J Immunopharmacol 17, 103-108 (1995).
37. H. C. Chen, F. M. Hofman, J. T. Kung, Y. D. Lin, B. A. Wu-Hsieh, Both virus and tumor necrosis factor alpha are critical for endothelium damage in a mouse model of dengue virus-Induced hemorrhage. J Virol 81, 5518-5526 (2007).
38. Y. T. Yen, H. C. Chen, Y. D. Lin, C. C. Shieh, B. A. Wu-Hsieh, Enhancement by tumor necrosis factor alpha of dengue virus-induced endothelial cell production of reactive nitrogen and oxygen species is key to hemorrhage development. J. Virol 82, 12312-12324 (2008).
39. M. Tauseef, N. Knezevic, K. R. Chava, M. Smith, S. Sukriti, N. Gianaris, A. G. Obukhov, S. M. Vogel, D. E. Schraufnagel, A. Dietrich, L. Bimbaumer, A. B. Malik, D. Mehta, TLR4 activation of TRPC6-dependent calcium signaling mediates endotoxin-induced lung vascular permeability and inflammation. J Exp Med 209, 1953-1968 (2012).
40. A. Uu, P. Gong, S. W. Hyun, K. Z. Wang, E. A. Cates, D. Perkins, D. D. Bannerman, A. C. Puche, V. Y. Toshchakov, S. Fang, P. E. Auron, S. N. Vogel, S. E. Goldblum, TRAF6 protein couples Toll-like receptor 4 signaling to Src family kinase activation and opening of paracellular pathway in human lung microvascular endothelia. J Biol Chem 287, 16132-16145 (2012).

1. A method for modulating production of a pro-inflammatory mediator (e.g., a cytokine) by a cell (e.g., an immune cell (e.g., a macrophage or monocyte), or an endothelial cell) in a subject with a Flavivirus infection, the method comprising, consisting or consisting essentially of contacting the cell with a pro-inflammatory mediator-modulating amount of a TLR4 antagonist.

2. (canceled)

3. (canceled)

4. (canceled)

5. (canceled)

6. A method according to claim 1, further comprising identifying that the subject has or is at risk of developing a Flavivirus infection, suitably prior to administration of the TLR4 antagonist.

7. A method according to claim 6, comprising determining the presence of NS1 (e.g., soluble or non-soluble forms of NS1) in the subject.

8. A method according to claim 7, comprising determining the presence of NS1 in a biological sample of the subject.

9. A method according to claim 8, wherein the biological sample is selected from blood, serum, plasma, saliva, cerebrospinal fluid, urine, skin or other tissues, or fractions thereof.

10. (canceled)

11. (canceled)

12. A method or use according to claim 1, wherein the Flavivirus is a virus selected from the group consisting of Dengue virus, Japanese encephalitis virus, Yellow fever virus, Murray Valley encephalitis virus, West Nile virus, Tick-borne encephalitis virus, St Louis encephalitis virus, Alfuy virus, Koutango virus, Cacipacore virus, and Yaounde virus.

13. A method or use according claim 12, wherein the Flavivirus is selected from Dengue virus serotype I, II, III, or IV.

14. A method or use according to claim 1, wherein the TLR4 antagonist is selected from nucleic acids, peptides, polypeptides, peptidomimetics, carbohydrates, lipids or other organic (carbon containing) or inorganic molecules.

15. A method or use according claim 14, wherein the TLR4 antagonist is selected from liposaccharide compounds, carbohydrates, small molecule inhibitors, nucleic acid molecules (e.g., ones that inhibit the transcription or translation of a TLR4 gene or that mediate RNA interference), decoy receptors and antagonist antibodies.

16. A method or use according to claim 1, wherein the TLR4 antagonist is represented by a structure according to any one of formulas (I), (II), (III), (IV), (V) and (VI), as defined herein above.

17. A method or use according to claim 1, wherein the TLR4 antagonist is a selective TLR4 antagonist.

18. A method or use according to claim 1, wherein the TLR4 antagonist is a non-selective TLR4 antagonist.

19. A method or use according to claim 1, wherein TLR4 antagonist is administered in combination with one or more ancillary agents that treat or ameliorate the symptoms of a Flavivirus infection.

20. A pharmaceutical composition, suitably for treating a Flavivirus infection or symptom thereof, comprising, consisting or consisting essentially of a TLR4 antagonist and an ancillary anti-Flaviviridae virus agent, optionally together with a pharmaceutically acceptable carrier or diluent.

21. A method for treating or preventing a Flavivirus infection or symptom thereof in a subject, the method comprising, consisting or consisting essentially of administering concurrently to the subject an effective amount of a TLR4 antagonist and an effective amount of an ancillary anti-Flavivirus agent.

22. A method or use according claim 21, wherein the TLR4 antagonist and the ancillary anti-Flavivirus agent are administered in synergistically effective amounts.

23. A method or use according claim 21, wherein the ancillary anti-Flavivirus agent is selected from interferons, illustrative examples of which include interferon alpha (e.g., interferon alpha 2a and interferon alpha 2b) and interferon beta (e.g., interferon beta 1a and interferon beta 1b), or nucleic acid constructs from which the ancillary anti-Flavivirus agent is expressible.
